(19) United States
(12) Patent Application Publication
(10) Pub. No.: US 2025/0388561 A1
(43) Pub. Date: Dec. 25, 2025

(54) A BICYCLOPENTYL THIANTHRENIUM COMPOUND, PROCESS FOR PREPARING THE SAME AND THE USE THEREOF

(71) Applicant: Studiengesellschaft Kohle gGmbH, Muelheim (DE)

(72) Inventors: Tobias RITTER, Muelheim (DE); Zibo BAI, Muelheim an der ruhr (DE); Eva Maria Wara ALVAREZ PARI, Evanston, IL (US)

(73) Assignee: Studiengesellschaft Kohle gGmbH, Muelheim (DE)

(21) Appl. No.: 18/879,405

(22) PCT Filed: Jun. 20, 2023
(86) PCT No.: PCT/EP2023/066609

§ 371 Date: Dec. 27, 2024

(30) Foreign Application Priority Data
Jun. 28, 2022 (EP) 22181685.3

(51) Int. Cl. C07D 339/08; C07C 41/09; C07C 209/74; C07D 213/61; C07D 221/28; C07D 231/12; C07D 231/38; C07D 263/24; C07D 319/06; C07D 401/12; C07D 471/04; C07D 487/04

(52) U.S. Cl. CPC C07D 339/08; C07C 41/09; C07C 209/74; C07D 213/61; C07D 221/28; C07D 231/12; C07D 231/38; C07D 263/24; C07D 319/06; C07D 401/12; C07D 471/04; C07D 487/04; C07C 2602/16

(57) ABSTRACT

The present inventions discloses a novel bicyclopentyl thianthrenium compound referred to as TT-BCP⁺X⁻, a process for preparing the same and the use thereof for bicyclopentylating organic compounds.

The present inventions refers a novel bicyclopentyl thianthrenium compound referred to as TT-BCP⁺X⁻, a process for preparing the same and the use thereof for bicyclopentylating organic compounds.

Bicyclopentanes (BCPs) are three dimensional isosteres of phenyl rings and, when 1,3-disubstituted, provide two exit vectors that are opposing each other, as in 1,4-disubstituted arenes. About 45% of marketed small molecule drugs contain phenyl substituents. Replacement of aryl substituents with 1,3-disubstituted bicyclopentanes can offer improvement of metabolic and pharmacokinetic properties of drug candidates; for example, the replacement of the fluorobenzene motif with BCP in the γ-secretase inhibitor BMS-708163 led to an increase of the aqueous solubility and metabolic stability over the parent compound as determined by in vivo mouse models. Currently, there are two main approaches to access 1,3-disubstituted BCPs within molecules of interest: Use of highly reactive [1.1.1]propellane as a starting material, which must be prepared before use because it has limited shelf stability even at −20° C., and functionalized BCPs for alkylation. Several impressive examples that employ [1.1.1]propellane, for example for N-alkylation or even difunctionalization, have been advanced over the recent past. Despite the large synthetic utility of the products, all the synthetic routes have in common that [1.1.1]propellane is not suitable for central production and distribution and therefore of attenuated utility for practitioners. A more practical reagent of similar or greater utility could increase the occurrences of BCP substituent incorporation to benefit from their desirable properties but such a reagent class has not yet been reported. Several useful BCP-based reagents such as Grignard reagents, iodides, boronates, and redox-active esters, have been reported in the prior art. Most such reagents can successfully engage in C—C bond formations, yet, none has reached the generality in reactivity of [1.1.1]propellane, and they often lack stability for storage or require multiple steps for preparation.

In the prior art, the reaction of bicyclopentane boronate with phenyl halides and N-nucleophiles has been shown. For example, SHELP RUSSELL A. ET AL discloses the synthesis and functionalization of benzylamine bicyclo[1.1.1]pentyl boronates in CHEMICAL SCIENCE, Vol. 12, No. 20, 2021, pages 7066-7072. Similarly, MASAKI KONDO ET AL: describes the silaboration of [1.1.1]propellane for providing a storable feedstock for bicyclo[1.1.1]pentane derivatives in ANGEWANDTE CHEMIB, Vol. 132, No. 5, 2019, pages 1986-1990. Furthermore, VANHEYST MICHAEL D. ET AL discloses the continuous flow-enabled synthesis of bench-stable bicyclo[1.1.1]pentane trifluoroborate salts and their utilization in metallaphotoredox cross-couplings” in ORGANIC LETTERS, Vol. 22, No. 4, 28 Jan. 2020 (Jan. 28, 2020), pages 1648-1654. The use of vinyl thianthrenium tetrafluoroborate for vinylating reactive species is generally disclosed in JACS, Vol. 143, No. 33, 2021, pages 12992-12998.

To date, no BCP-based reagents nor [1.1.1]propellane-based reactivity are available for aryl BCP ether synthesis.

In nature, sulfonium salts can act as efficient alkylation reagents. Similarly, chemists have made use of alkylation reactions based on sulfonium salts but the transfer of tertiary alkyl groups, such as bicyclopentyl, remains unknown. The inventors have previously reported new reactivity of arylthianthrenium (TT) salts that can expand the chemical space in comparison to other (pseudo)halides and be rationalized by the unusual properties of the thianthrene scaffold. Based on the single electron reactivity, the high reduction potential, and the ability to function as good leaving group and readily engage in radical chemistry, the inventors devised a synthesis of BCP-thianthrenium salts to function as readily available, stable, and versatile alkylating reagents. Thianthrenium-substituted BCPs can engage in radical chemistry, distinct from that of conventional alkyl sulfonium salts, that productively combines photoredox catalysis with transition-metal-mediated bond formation. While copper catalysis has been productive for thianthrene- and BCP-based chemistry, the inventors also introduce here previously unreported photoredox mediated nickel catalyzed cross-coupling with thianthrenium salts.

Because of its simple preparation, handling, high reactivity, and broad tolerance of functional groups present in complex molecules, as well as its divergent reactivity, the inventors expect that TT-BCP⁺X⁻, in particular TT-BCP BF₄⁻ will find widespread utility in future reaction chemistry development. The main difference of the inventive TT-BCP⁺X⁻ when compared to most other sulfonium-based reagents is its simple one-step synthesis protocol; in contrast, the practical synthesis for the classical Umemoto's reagent requires nine steps. The fundamental difference to the Togni reagents is the higher reduction potential of the inventive TT-CF₃⁺X⁻, a consequence of the positive charge that can result in complementary reactivity in single electron transfer reactions when compared to the λ³-iodane compounds.

Thus, the present invention is directed to an optionally substituted bicyclopentyl thianthrenium derivative TT-BCP⁺X⁻. In more detail, the present invention is thus directed, in a first aspect, to a thianthrene derivative of the Formula (I):

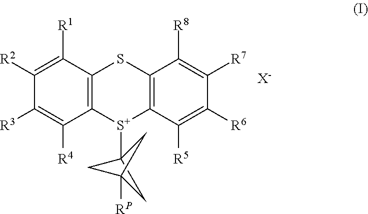

wherein R¹ to R⁸ may be the same or different and are each selected from hydrogen, halogen, a C₁ to C₆ alkyl group, which is optionally substituted by at least one halogen, or a —O—C₁ to C₆ alkyl group, wherein R^{P} represents CF₃ or CN, and wherein X⁻ is an anion, selected from F⁻, Cl⁻, triflate⁻, BF₄⁻, SbF₆⁻, PF₆⁻, ClO₄⁻, 0.5 SO₄²⁻ or NO₃⁻.

In an embodiment of the thianthrene derivative of the Formula (I), R¹ to R⁸ may be the same or different and are each selected from hydrogen, Cl or F, R^{P} is as defined in claim 1 and X⁻ is an anion as defined before, preferably triflate, or BF₄.

In another embodiment of the thianthrene derivative of the Formula (I), R², R³, R⁶ and R⁷ are selected from —OCH₃, F or CF₃ and the others of R¹ to R⁸ are hydrogen, R^{P} is as defined in claim 1 and X⁻ is an anion as defined before, preferably a triflate or BF₄ anion.

In yet another embodiment of the thianthrene derivative of the Formula (I), R¹ to R⁸ are hydrogen, R^{P} is as defined in claim 1 and X⁻ is an anion as defined before, preferably a triflate or BF₄ anion.

In the above formulae, X⁻ represents an anion selected from F⁻, Cl⁻, triflate⁻, BF₄⁻, SbF₆⁻, PF₆⁻, ClO₄⁻, 0.5 SO₄²⁻, or NO₃⁻, and similar anions which result in a stable ion pair with the bicyclopentyl thianthrenium cation.

The inventive TT-BCP⁺X⁻ is useful for bicyclopentylating organic compounds selected from aryl halides, phenols, and nucleophilic compounds including N-nucleophiles. Thus, the present invention is furthermore directed, in a second aspect, to the use of a bicyclopentyl thianthrenium compound of the Formula (I) as a transfer agent for transferring a bicyclopentyl group to an organic compound selected from aryl halides, phenols, and nucleophilic compounds including N-nucleophiles, which is optionally substituted by at least group selected from hydroxyl, aldehyde, carboxylic acid ester, olefin, amino, amido, sulfonamido, halogen such as bromo, fluoro, chloro,

Thus, the inventive bicyclopentyl thianthrenium compound of the Formula (I) can be used as a transfer agent for transferring a bicyclopentyl group under irradiation in the presence of a photocatalyst in a transition-metal-mediated bond formation to an organic compound selected from aryl halides, phenols, and nucleophilic compounds including N-nucleophiles which reaction includes alkylation of phenols, N-heterocycles, amines, amides, sulfonamides, anilines, arenes, heteroarenes and haloarenes.

In the inventive process for preparing the inventive TT-BCP⁺X⁻ or the use thereof for bicyclopentylating, the choice of the organic solvent is not critical as long as it is an aprotic organic solvent selected from acetonitrile, other nitriles, chlorinated hydrocarbons, or other aprotic solvents, or mixtures thereof. The reaction conditions are also not critical and the reaction is usually carried out at a temperature between −78° C. and 50° C., preferably 0° C. to 30° C., under ambient pressure and optionally under an inert gas atmosphere.

In the inventive process for transferring the bicyclopentyl group, the organic compound may be a monocyclic or polycyclic, aromatic or heteroaromatic hydrocarbon ring structure having 5 to 22 carbon atoms, which may be unsubstituted or substituted by one of more substituents selected from saturated or unsaturated, straight chain or branched aliphatic hydrocarbons having 1 to 20 carbon atoms, aromatic or heteroaromatic hydrocarbons having 5 to 22 carbon atoms, heterosubstituents, or which may be part of a cyclic hydrocarbon ring system (carbocyclic) with 5 to 30 carbon atoms and/or heteroatoms. The definition for said aliphatic hydrocarbons may include one or more heteroatoms such O, N, S in the hydrocarbon chain.

In the context of the aspects of the present invention, the following definitions are more general terms which are used throughout the present application.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The term “aliphatic” includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “lower alkyl” is used to indicate those alkyl groups (cyclic, substituted, unsubstituted, branched or unbranched) having 1-6 carbon atoms.

As used herein, “alkyl” refers to a radical of a straight-chain, branched or cyclic saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

“Aryl” or aromatic hydrocarbon refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” or heteroaromatic hydrocarbon refers to a radical of a 5-14 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Unsaturated” or “partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups, are further referred to using the suffix -ene, e.g., alkylene, alkenylene, alkynylene, carbocyclylene, heterocyclylene, arylene, and heteroarylene.

An atom, moiety, or group described herein may be unsubstituted or substituted, as valency permits, unless otherwise provided expressly. The term “optionally substituted” refers to substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. For purposes of this invention, heteroatoms such as nitrogen may have hydrogen substituents and/or any suitable substituent as described herein which satisfy the valencies of the heteroatoms and results in the formation of a stable moiety. In certain embodiments, the substituent is a carbon atom substituent. In certain embodiments, the substituent is a nitrogen atom substituent. In certain embodiments, the substituent is an oxygen atom substituent. In certain embodiments, the substituent is a sulfur atom substituent.

Exemplary substituents include, but are not limited to, halogen, —CN, —NO₂, —N₃, —SO₂H, —SO₃H, —OH, —O-alkyl, —N-dialkyl, —SH, —S.alkyl, —C(═O)alkyl, —CO₂H, —CHO.

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

The term “catalysis,” “catalyze,” or “catalytic” refers to the increase in rate of a reaction due to the participation of a substance called a “catalyst.” In certain embodiments, the amount and nature of a catalyst remains essentially unchanged during a reaction. In certain embodiments, a catalyst is regenerated, or the nature of a catalyst is essentially restored after a reaction. A catalyst may participate in multiple chemical transformations. The effect of a catalyst may vary due to the presence of other substances known as inhibitors or poisons (which reduce the catalytic activity) or promoters (which increase the activity). Catalyzed reactions have a lower activation energy (rate-limiting free energy of activation) than the corresponding uncatalyzed reaction, resulting in a higher reaction rate at the same temperature. Catalysts may affect the reaction environment favorably, or bind to the reagents to polarize bonds, or form specific intermediates that are not typically produced by a uncatalyzed reaction, or cause dissociation of reagents to reactive forms.

The invention is not intended to be limited in any manner by the above exemplary listing of substituents.

In conclusion, the inventors have developed a new bicyclopentylating reagent, bicyclopentyl thianthrenium triflate (3, TT-BCP⁺ BF₄⁻), which is easily accessible from readily available starting materials in a single step. The new reagent can engage in various reactions and promises to be of synthetic utility.

The invention is further illustrated by the attached Figures and Examples. In the Figures, the respective Figure shows:

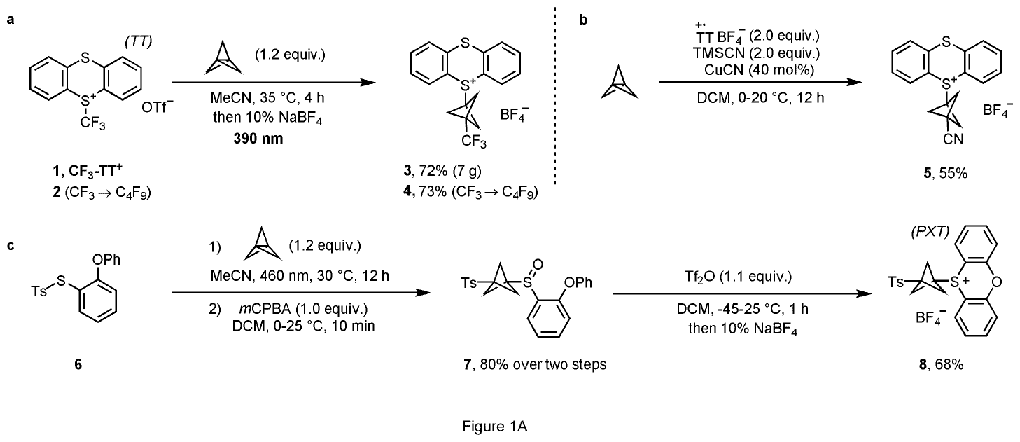

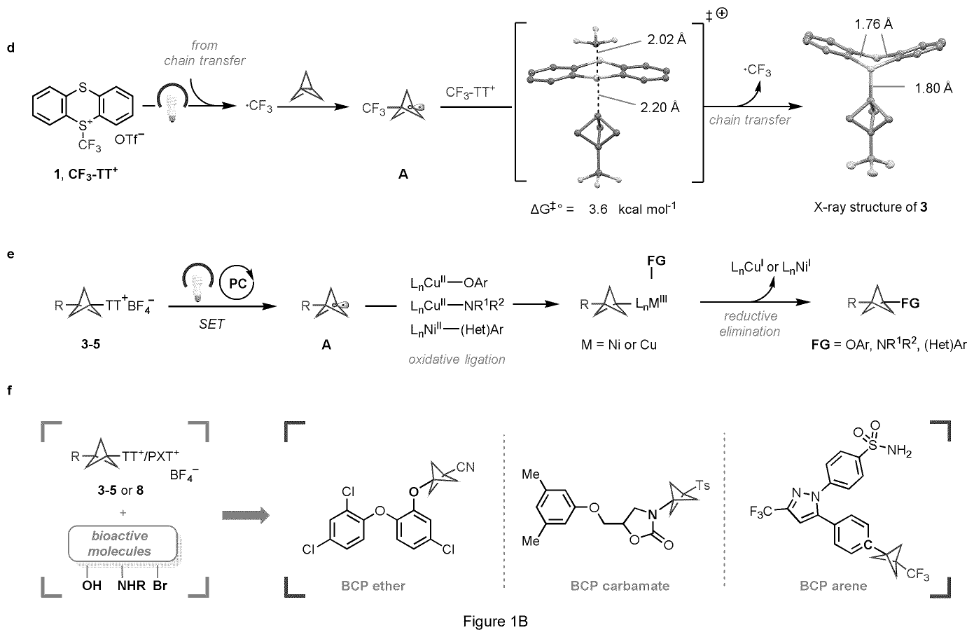

FIGS. 1A and 1B: Bicyclo [1.1.1]pentane thianthrenium salts;

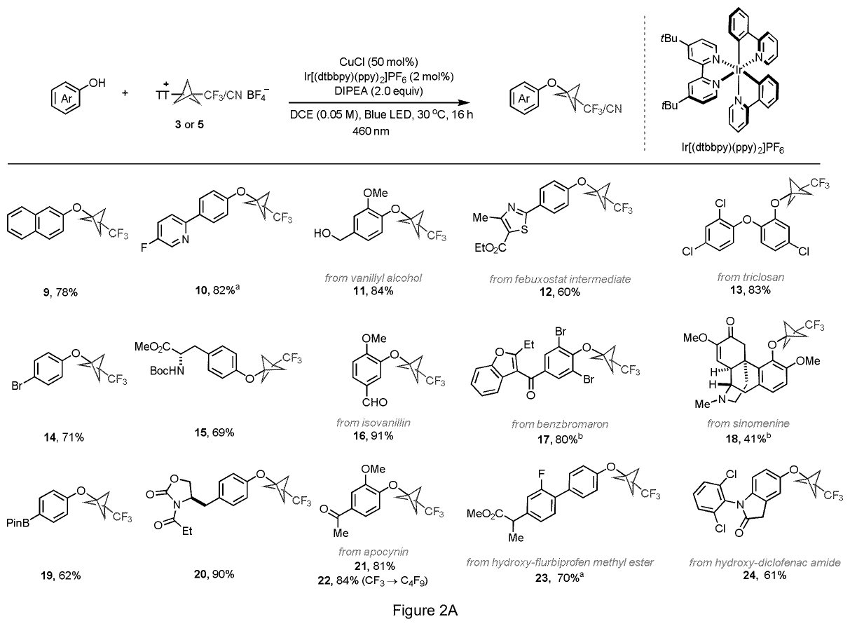

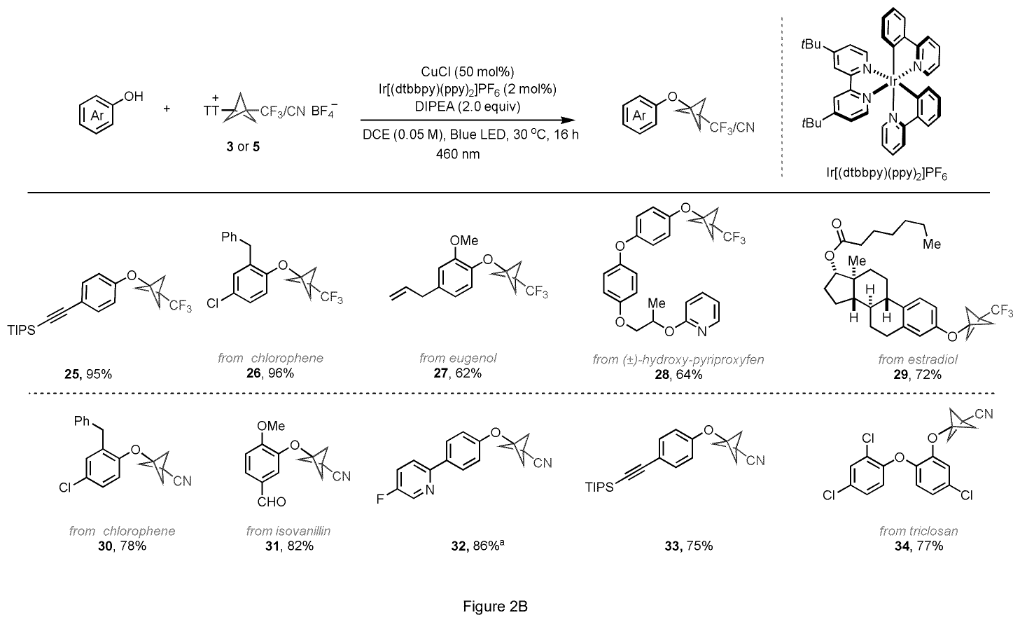

FIGS. 2A and 2B: Substrate scope for Cu-catalyzed C—O cross coupling of 3, 4, and 5 with phenols;

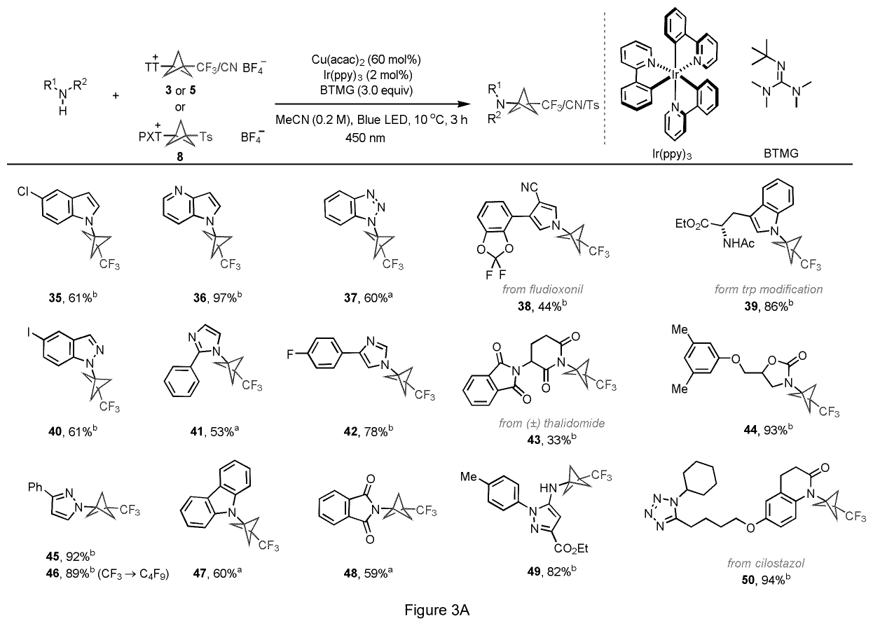

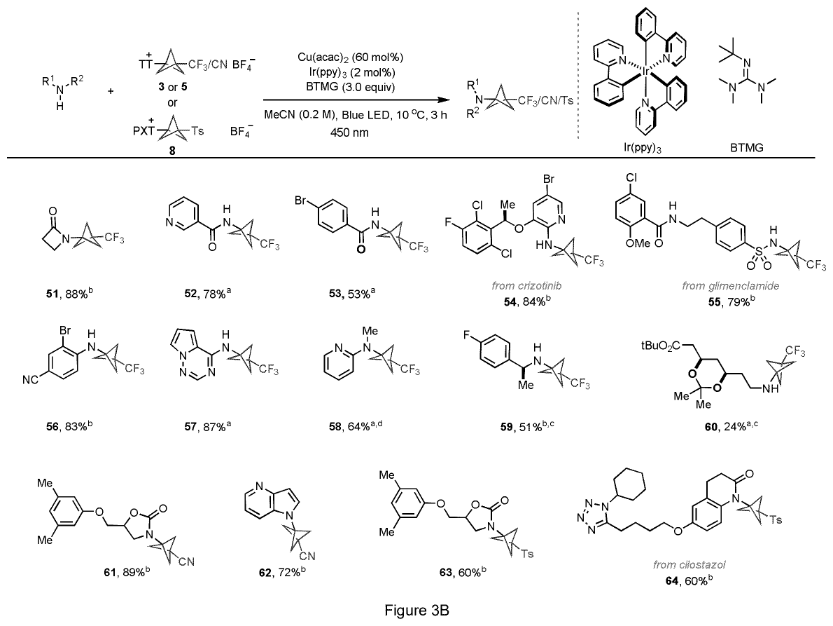

FIGS. 3A and 3B: Substrate scope for C—N cross coupling of 3, 4, 5, and 8 with N-nucleophiles;

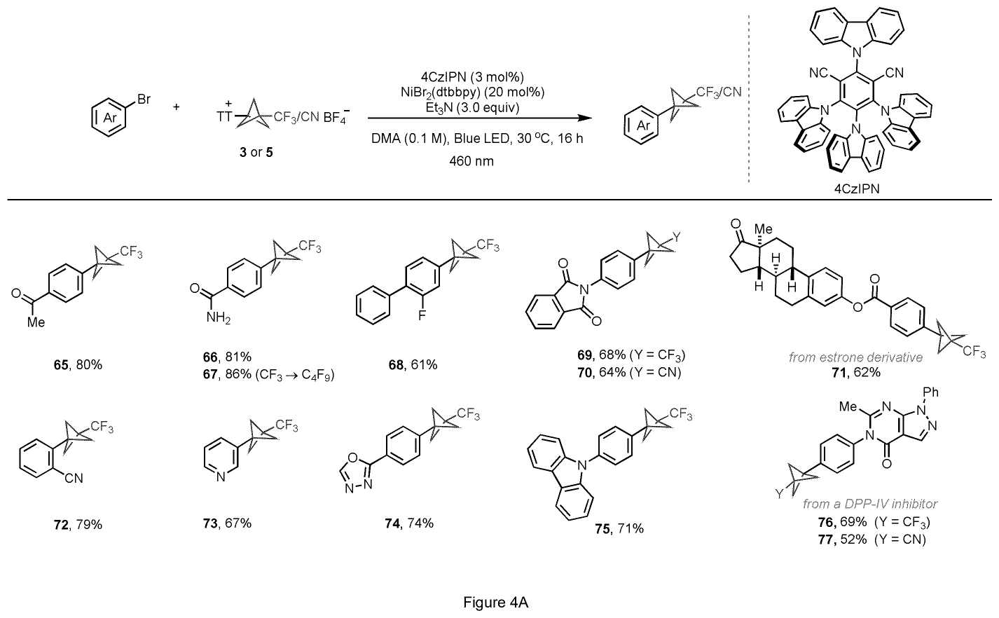

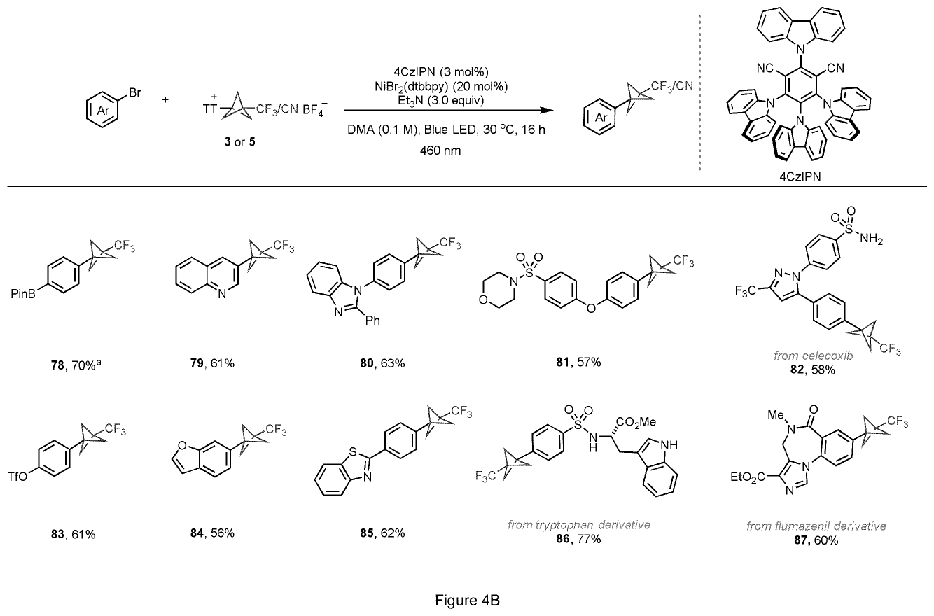

FIGS. 4A and 4B: Substrate scope for Ni-catalyzed reductive C—C cross coupling of 3, 4, and 5 with (het)aryl bromides.

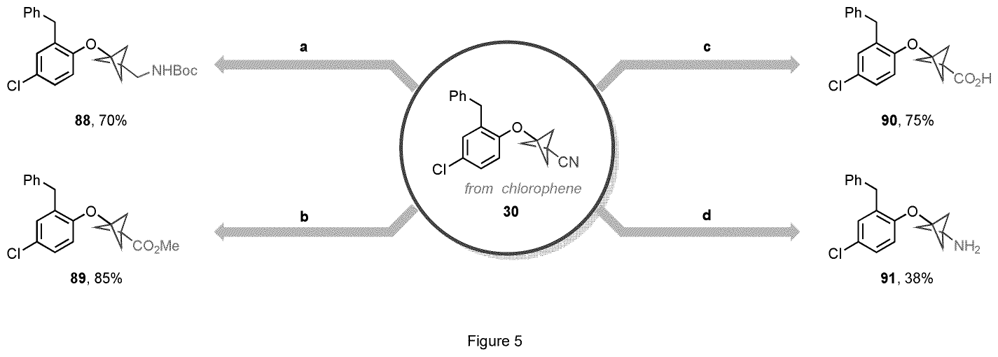

FIG. 5: Synthetic utility of the methodology.

In more detail, FIG. 1 illustrates:
a: Synthesis of CF₃BCP-TT⁺ BF₄⁻ (3) and ⁿC₄F₉BCP-TT⁺ BF₄⁻ (4) reagents. The general reaction conditions are as follows: CF₃-TT⁺ OTf⁻ (1.0 equiv), [1.1.1]propellane (1.2 equiv.), MeCN (0.14 M), purple LED (390 nm), 35° C., 4 h, followed by aqueous workup with NaBF₄ (10% w/w).
b: Synthesis of CNBCP-TT⁺ BF₄⁻ (5) salt. Conditions: [1.1.1]propellane (1.0 equiv.), thianthrenium tetrafluoroborate (TT^{⋅+} BF₄⁻) (2.0 equiv.), TMSCN (2.0 equiv.), CuCN (40 mol %), DCM (0.20 M), 0-20° C., 12 h.
c: Synthesis of TsBCP-PXT⁺ BF₄⁻ (8) salt. Conditions: 1) 6 (1.0 equiv), [1.1.1]propellane (1.2 equiv.), MeCN (0.12 M), blue LED (460 nm), 30° C., 12 h; 2) mCPBA (1.0 equiv), DCM (0.3 M), 0-25° C., 10 min; 3) Tf₂O (1.1 equiv.), DCM (0.12 M), −45-25° C., 1 h.
d: Proposed mechanism of 3 formation proceeding through a radical chain propagation and crystal structure of 3. Thermal ellipsoids are drawn at the 50% probability level. Hydrogen atoms and counterion have been omitted for clarity.
e: Proposed mechanism for the cross-coupling of BCP-TT⁺ salts enabled by the photoredox catalysis and transition metal catalysis.
f: Synthetic utility of BCP-TT⁺/PXT⁺ salts. TT, thianthrene; CF₃, trifluoromethyl; TMS, trimethylsilyl; Ts, 4-toluolsulfonyl; PXT, phenoxathiin; PC, photoredox catalyst; Ar, aryl; Het, hetero; BCP, bicyclo[1.1.1]pent-1-yl.

As shown in the Figures, a practical synthesis of the CF₃BCP-TT⁺ salt 3 was accomplished by an addition reaction between the trifluoromethylthianthrenium reagent 1 and [1.1.1]propellane (FIG. 1a). All experimental observations and DFT calculations are consistent with a radical chain transfer mechanism that includes irradiation of 1 with purple LEDs at a wavelength transparent to 3 to induce homolytic S—CF₃ cleavage, followed by radical addition of CF₃ radical to [1.1.1]propellane to form putative BCP radical A. Subsequent chain propagation by TT radical cation abstraction from 1 by A through a low lying transition state TS is supported by DFT and consistent with a measured quantum yield of ϕ=16 (FIG. 1d). The stability of the TT radical cation sets thianthrene apart from conventional sulfides. In contrast to volatile and thermally unstable propellane, compound 3 is a non-hygroscopic, free-flowing powder that can be stored under ambient conditions without observable decomposition for at least one year, and a melting point of 150° C. A differential scanning calorimetry (DSC) analysis confirmed that heating of 3 up to 170° C. is not accompanied by any exothermic decomposition process, which attests to its favorable safety properties. Compound 3 is made from synthetically involved [1.1.1]propellane, yet, the practitioner interested in BCP substitution would not be required to handle the unstable reagent if 3 were produced centrally and distributed. Based on the same strategy, we prepared nonafluorobutyl BCP-TT⁺ 4 from 2 in 73% yield. In addition, a synthesis other than radical chain transfer from S-substituted thianthrene-based reagents can access the novel compound class, as shown by a synthesis from the persistent thianthrenium radical cation to afford the cyano-substituted BCP reagent 5 (FIG. 1b) and a stepwise synthesis of 4-toluolsulfonyl BCP-phenoxathiin (PXT) reagent 8 starting from thiosulfonate 6 (FIG. 1c). It is expected that the cationic BCP-TT⁺ 3-5 are easily reduced by a single electron either by the excited state of a photoredox catalysts or by a reduced photoredox catalyst obtained through reductive quench from the excited state. We have observed reductive quenching of the excited photocatalyst Ir[(dtbbpy)ppy₂]PF₆ by Stern-Volmer quenching studies, for example in the presence of copper (I) to generate putative Ir(II) for SET reduction of 3 (E_{1/2} of 3=−1.4 V; [Ir^{III}/Ir^{II}]=−1.5 V, both versus SCE in MeCN. The ensuing chemoselective mesolytic cleavage of the exocyclic BCP-thianthrene C—S bond can be rationalized by both a significantly longer exocyclic C—S bond when compared to the endocyclic C—S bonds within the TT scaffold as determined by X-ray crystallographic analysis of 3 (FIG. 1d), and barrierless homolysis of the exocyclic C—S bond upon single electron reduction of 3 as supported by DFT. The resulting synthetically useful BCP radical is thus readily available in situ by functional-group-tolerant photoredox-mediated SET. Oxidative ligation of the BCP radical to transition metals in medium oxidation states, such as Cu(II) obtained through the reductive quench from the excited photocatalyst, or Ni(II) obtained from oxidative addition into aryl halides, can access high-valent transition metal BCP complexes. Ensuing facile reductive elimination reactions to attach the BCP scaffold to several atoms should be achievable from such high-valent complexes (FIG. 1e).

As shown in FIG. 2, the general reaction conditions for Cu-catalyzed C—O cross coupling of 3 or 5 with phenols are as follows: phenol (0.15 mmol, 1.0 equiv), 1 or 2 (2.0 equiv.), Ir[(dtbbpy)(ppy)₂]PF₆ (2 mol %), CuCl (50 mol %), DIPEA (2.0 equiv.), DCE (0.05 M), blue LED (40 W), 30° C., 16 h. ^{a}DCM (0.05 M) was used instead. ^{b}1 (3.0 equiv.) and CuCl (100 mol %) were used.

Aryl bicyclo[1.1.1]pentyl ethers have potential as bioisosteres for diaryl ether derivatives that are common structural motifs in natural and synthetic pharmaceutically important compounds. However, no synthesis to construct aryl BCP ethers is currently documented. Reagents 3-5 can successfully be employed in the metallaphotoredox-catalyzed alkylation of phenols with sub-stoichiometric amounts of copper salts to access the previously unknown class of aryl BCP ethers (Table 1). The reactions exhibits broad scope, and proceeds efficiently with phenols bearing electron-neutral, -rich, and -poor substituents (e.g. 9, 11, and 17, respectively), as well as ortho-substituted phenols (e.g. 13, 16, 18). Synthetically useful functional groups such as hydroxy (11), ester (12, 15, 23, 29), amide (15), aldehyde (16), 2-oxazolidone (20), ketone (21), lactam (24), alkynyl (25), alkenyl (27), and even tertiary amines (18) are tolerated, highlighting the mildness of the reaction conditions. Aryl chlorides (13, 24, 26) and bromides (14, 17) are tolerated, resulting in potential reactive sites for functional group interconversion. Similarly, Bpin (19) and TIPS (25) groups are tolerated, which are well known as nucleophilic coupling partners for Suzuki and Hiyama cross coupling, respectively. In addition, Lewis basic heterocycles, including pyridine (10) and thiazole (12), that can be a liability in transition metal catalyzed coupling reactions, do not inhibit the desired cross coupling reactivity. The reaction is chemoselective with respect to N-nucleophiles (e.g. 15, vide infra). Due to the large functional-group compatibility, late-stage functionalization of drug molecules, such as triclosan (13), benzbromaron (17), sinomenine (18), and chlorophene (26) are accessible. Combined with thianthrene-mediated late-stage aromatic C—H hydroxylation, we have realized a multistep site selective C—H/bicyclopentyloxylation of small-molecule pharmaceuticals and pesticides, such as flurbiprofen methyl ester (23), diclofenac amide (24), and pyriproxyfen (28).

As illustrated in FIG. 3, an analogous strategy was successful for bicyclopentylation of N-nucleophiles (Table 2). Distinct from published procedures for N-alkylation reactions with propellane, bicyclopentylation with 3-5 and 8 proceeds with a larger scope with respect to the nitrogen nucleophile. Medicinally relevant sub-structures such as indoles (35, 39) and pyrrole (38) are compatible with our protocol, as are 4-azaindole (36), benzotriazole (37), indazole (40), imidazoles (41, 42), pyrazoles (45, 46), and carbazole (47). Moreover, the methodology is not limited to N-heterocycles, examples of phtalimide (48), dihydroquinolinone (50), β-lactam (51), amides (52, 53), and sulfonamide (55) work well in this transformation. Aniline (56), 2-aminopyridines (54, 58), and 5-amino pyrazole (49) can also undergo C—N coupling in good yields. Notably, 2-aminopyrrolo[2,1-f][1,2,4]triazine (57) which is found in the structure of remdesivir (against COVID-19) can be functionalized efficiently. By slightly modifying the reaction conditions, the scope could be further extended to benzylic amines (59) and alkyl amines (60). As in the corresponding ether bond formation, large functional group tolerance, even for redox-active aryl iodides (40), enables late-stage modification of various pharmaceutically relevant molecules in drug discovery process (38, 39, 43, 50, 54, 55) as shown in Table 2. Basic, electron-rich tertiary amines are not tolerated, potentially a consequence of their single electron oxidation by excited photoredox catalysts. When more than one nitrogen nucleophile is present, functionalization of the more acidic position proceeds chemoselectively (e.g. 55). Both C—O and C—N bond forming reactions are, in principle, catalytic in transition metal, yet, use of about half an equivalent of copper afforded the highest yields. Although reduction of the copper loading is possible, the lower yield is, in our opinion, not justifiable given the low cost of the simple copper salts when compared to the cost of the other complex starting materials employed in these transformations.

In addition to its simple synthesis and stability, reagents 3-5 can, beyond C-heteroatom cross coupling with copper, also participate in metallophotoredox catalysis with nickel catalysts for reductive C—C cross coupling reactions with (het)aryl bromides (FIG. 4). Synergistic cooperation of nickel catalysis and photoredox catalysis with thianthrenium salts has not been reported before. Carbon-carbon cross coupling reactions of iodo-BCPs, BCP Grignard reagents, BCP-boronates, and BCP redox active esters have been developed previously but not with a reagent as synthetically convenient as 3-5. A mechanism from 3-5 could proceed through a Ni(0-II-III-I) cycle with oxidative ligation of the BCP radical to a Ni(II) aryl complex obtained by oxidative addition of Ni(0) to an aryl bromide, with ensuing reductive C—C elimination from a putative high-valent Ni(III) complex. The cross-coupling of electron poor arenes (65, 66, 72, 83) was successful; engaging electron-rich arenes resulted in lower yields (81). Under the current reaction conditions, a variety of functional groups could be tolerated such as ketones (65), amides (66, 67), esters (71, 86, 87), nitriles (72), heteroarenes (74, 76, 80, 82, 85-87), and 1°-3° sulfonamides (81, 82, 86). The reactive functional groups Bpin (78), and triflate (83) are also well tolerated. The synthetic utility of the strategy was further exemplified by the functionalization of heteroaromatic bromides (73, 79, 84) and pharmaceuticals (77, 82, 87). The most prominent side reaction for electron-rich aryl bromides is proto-debromination.

As shown in FIG. 4, the general reaction conditions are as follows: Aryl halide (0.20 mmol, 1.0 equiv), 3-5 (1.5 equiv.), 4CzIPN (3 mol %), Ni(dtbbpy)Br₂ (20 mol %), Et₃N (3.0 equiv.), DMA (0.1 M), blue LED (460 nm, 40 VA, 30° C., 16 h. ^{a}Ni(dtbbpy)Cl₂ (20 mol %) was used as catalyst.

To highlight the synthetic utility of the methodology, the inventors performed several transformations on cyanobicyclo[1.1.1]pentylether 30 (FIG. 5). For example, reduction of 30 with NiCl₂ and NaBH₄ afforded alkyl amine 88 in 70% yield. In addition, the cyano group was converted to BCP ester 89 and BCP carboxylative acid 90. Finally, BCP amine 91 was prepared from the 30 via Curtius rearrangement.

As shown in FIG. 5, the synthetic transformations of cyanobicyclo[1.1.1]pentylether 30 are as follows: a) NiCl₂, NaBH₄, Boc₂O, MeOH, 25° C., 6 h. b) H₂SO₄, MeOH, 65° C., 12 h. c) 1) H₂SO₄, MeOH, 65° C., 12 h; 2) LiOH·H₂O, THF/H₂O, 25° C., 3 h. d) 1) H₂SO₄, MeOH, 65° C., 12 h; 2) LiOH·H₂O, THF/H₂O, 25° C., 3 h; 3) DPPA, Et₃N, toluene, 25-105° C., 6 h, then 1M HCl, 60° C., 12 h.

The inventors report a storable, thianthrenium-based class of BCP-transfer reagents that can afford potentially valuable small molecules that are in part currently inaccessible by other methods. The inventors anticipate that commercial availability of a stable and readily employed reagent would enable practitioners, for example in the pharmaceutical industry, to introduce the promising BCP substituent substantially more straightforwardly into small molecules of interest than is possible today.

### Materials and Methods

All reactions were carried out under an ambient atmosphere unless otherwise stated and monitored by thin-layer chromatography (TLC). Air- and moisture-sensitive manipulations were performed using standard Schlenk- and glove-box techniques under an atmosphere of argon or dinitrogen. High-resolution mass spectra were obtained using Q Exactive Plus from Thermo. Concentration under reduced pressure was performed by rotary evaporation at 25-40° C. at an appropriate pressure. Purified compounds were further dried under high vacuum (0.010-0.005 mBar). Yields refer to purified and spectroscopically pure compounds, unless otherwise stated.

### Solvents

Anhydrous DCE and DMA were purchased from Acros Organics and Sigma Aldrich. Other anhydrous solvents were obtained from Phoenix Solvent Drying Systems. All deuterated solvents were purchased from Euriso-Top.

### Chromatography

Thin layer chromatography (TLC) was performed using EMD TLC plates pre-coated with 250 μm thickness silica gel 60 F₂₅₄ plates and visualized by fluorescence quenching under UV light and KMnO₄ stain. Flash column chromatography was performed using silica gel (40-63 μm particle size) purchased from Geduran®.

### Photochemistry

All N-alkylation reactions with blue light were carried out using a photoreactor equipped with a blue LED module (KT-Elektronik, “100 W Power LED blau 450 nm Aquarium”, 450 nm, 100 W), consisting out of 100 LED-chips. The power of the LED was adjusted using a linear regulator. The vials were cooled with two Peltier-elements (TEC1-12706) while being irradiated with blue light.

### Spectroscopy and Instruments

NMR spectra were recorded on a Bruker Ascend™ 500 spectrometer operating at 500 MHz, 471 MHz and 126 MHz, for ¹H, ¹⁹F and ¹³C acquisitions, respectively; or on a Varian Unity/Inova 600 spectrometer operating at 600 MHz and 151 MHz for ¹H and ¹³C acquisitions, respectively. Chemical shifts are reported in ppm with the solvent residual peak as the internal standard.

### Starting Materials

All substrates were used as received from commercial suppliers, unless otherwise stated. The [1.1.1]propellane solution was prepared according to the literature. Trifluoromethyl thianthrenium triflate salt (CF₃-TT⁺ OTf⁻) was prepared according to the literature. Thianthrene radical cation (TT^{⋅+}) was prepared according to the literature. Ir(ppy)₃ was purchased from Sigma-Aldrich. Ir[(dtbbpy)(ppy)₂]PF₆ was purchased from Sigma-Aldrich. CuCl was purchased from Alfa Aesar. Et₃N was purchased from Acros Organics. Ni(dtbbpy)Br₂ was prepared according to the literature. 4CzIPN was prepared according to the literature. The phenols and aryl bromides were prepared according to the literature.

### Experimental Part

### Preparation of [1.1.1]Propellane Stock Solution

This compound was prepared following the procedure reported by Anderson:

To an oven-dried 500 mL round-bottom flask containing a teflon-coated magnetic stirring bar was added 1,1-dibromo-2,2-bis(chloromethyl)cyclopropane (16.0 g, 52.8 mmol, 1.00 equiv.). The flask was sealed with a septum-cap, evacuated, and back-filled with argon three times, and then anhydrous Et₂O (33 mL) was added. The mixture was cooled to −45° C. (dry ice/isopropanol bath). Phenyllithium (56 mL, 1.9 M in Bu₂O, 0.11 mol, 2.0 equiv.) was added dropwise via syringe over 15 min at −45° C. The cooling bath was replaced with an ice bath, and the mixture was warmed to 0° C., and then stirred at this temperature for 2 h.

Upon completion of the reaction, the mixture was then distilled at 25° C. (70 mbar) using a rotary evaporator with dry ice trap, the receiving flask of which was immersed in a dry ice/acetone bath. The [1.1.1]propellane stock solution (31 mL, 0.85 M in Et₂O, 50%) was transferred to a flame-dried septum-sealed bottle under an inert atmosphere, and stored at −20° C. The approximate concentration of the solution was determined by quantitative ¹H NMR spectroscopy with 1,2-dichloroethane as an internal standard.

### Quantitative NMR Experiment

A sample of the solution containing [1.1.1]propellane in diethyl ether (100 μL) was diluted with dichloroethane (DCE) (25 μL) and CDCl₃ was added (ca. 0.5 mL). The ratio of the DCE:propellane was determined and used for the calculation of the concentration of the propellane solution. This was performed in duplicate and the average of the two runs was used as the final approximated concentration.

### Preparation of bicyclo[1.1.1]pentyl thianthrenium salts

### Trifluoromethylbicyclo[1.1.1]pentyl thianthrenium salt (3)

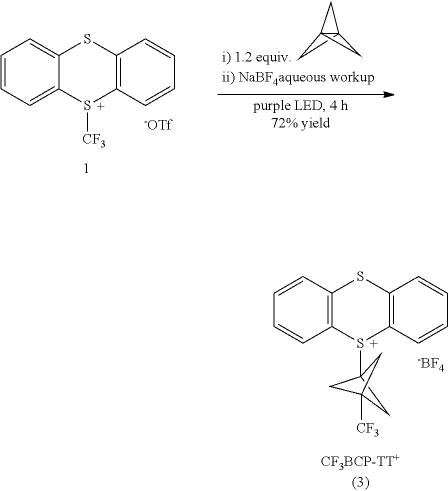

To a 500 mL round-bottom flask equipped with a stirring bar were added trifluoromethyl thianthrenium salt 1 (9.23 g, 21.2 mmol, 1.00 equiv.) and anhydrous MeCN (152 mL, 0.140 M). The flask was capped with a rubber septum, and subsequently [1.1.1]propellane solution in Et₂O (c=0.85 M, 30 mL, 1.7 g, 1.2 equiv.) was added dropwise via syringe to the reaction over 10 min while stirring. Subsequently, the reaction flask was placed 11 cm away from two Kessil PR160-390 nm LEDs. The mixture was irradiated for 4 h while maintaining the temperature at approximately 35° C. through cooling with a fan. After 4 h, the reaction flask was removed from the two Kessil PR160-390 nm LEDs. The mixture was concentrated under reduced pressure, diluted with DCM (150 mL). The DCM solution was poured into a separation funnel and washed with aqueous NaBF₄ solution (3×ca. 150 mL, 10% w/w). All organic phases were combined, dried over MgSO₄ (10 g), filtered, and the solvent evaporated under reduced pressure. The crude material was purified by chromatography on silica gel eluting first with 100% EtOAc and later DCM/i-PrOH (1/0-95/5 (v/v)) to afford the title compound as a brown solid. The solid material was dissolved in DCM (ca. 30 mL), and the flask containing the solid in DCM was placed in an ice bath, at which point the residue was triturated with Et₂O (ca. 200 mL). The flask was kept at 0° C. for 1 h. The resulting solid was decanted and washed with ice-cold Et₂O (2×ca. 50 mL), and dried under vacuum overnight yielding a beige solid 3 (6.73 g, 15.3 mmol, 72%).

$${R}_{f} = 0.53 ( DCM / MeOH , 10 : 1 ( v / v ) ) .$$

Melting point: 150-151° C. (recryst. solvents: DCM/Et₂O (2:1)). The melting process is accompanied by decomposition.

Elemental analysis calcd (%) for C₁₈H₁₄BF₇S₂: C, 49.33, H, 3.22; found: C, 49.09, H, 3.21.

### Nonafluorobutyl Thianthrenium Salt (2)

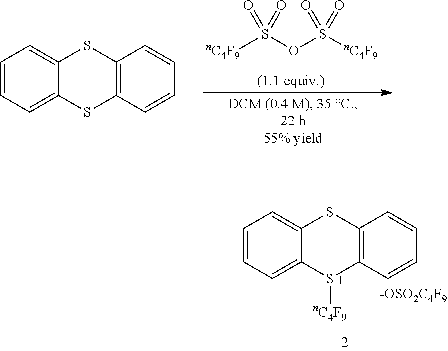

Under an ambient atmosphere, a 25 mL two-neck round bottom flask equipped with a teflon-coated magnetic stirring bar, was charged with thianthrene (647 mg, 3.12 mmol, 1.00 equiv.) and DCM (8 mL, c=0.4 M). Subsequently, nonafluorobutanesulfonic anhydride (2.0 g, 1.1 mL, 3.4 mmol, 1.1 equiv.) was added in one portion at 25° C. Upon addition of anhydride, the reaction mixture rapidly turned light purple and gradually deepened, accompanied by formation of suspended particles. The reaction mixture was stirred at 35° C. for 22 h. Subsequently, a saturated aqueous NaHCO₃ solution (ca. 5 mL) was added carefully. At this point, the purple color faded away, and the suspension turned light brown. The suspension was poured into a 50 mL separatory funnel, and the aqueous layer was discarded. The organic layer was concentrated to dryness under reduced pressure, resulting in the formation of a light brown residue. Diethyl ether (10 mL) was added to the residue and the suspension was stirred vigorously at 25° C. for 30 min. The mixture was allowed to stand for 5 min, subsequently, the solvent was decanted carefully. In order to obtain an analytically pure compound, the decanting process was repeated there times with diethyl ether. The resulting yellow slurry was concentrated to dryness under reduced pressure to afford 2 (1.3 g, 1.7 mmol, 55%) as a pale yellow solid.

$${R}_{f} = 0.36 ( DCM / MeOH , 10 : 1 ( v / v ) ) .$$

Melting point: 125-126° C.

HRMS-ESI (m/z) calculated for C₁₆H₈S₂F₉⁺ [M-OSO₂C₄F₉]⁺, 434.9919; found, 434.9918; deviation: −0.3 ppm.

### Nonafluorobutylbicyclo[1.1.1] pentyl thianthrenium salt (4)

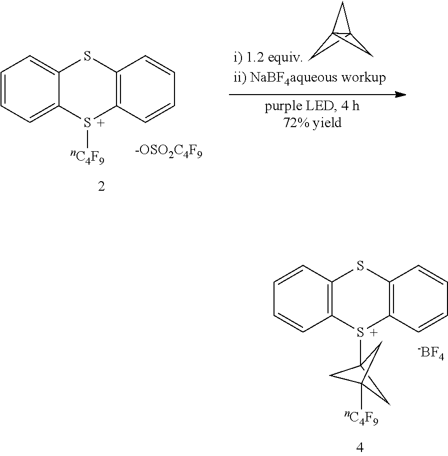

To a 25 mL round-bottom flask equipped with a stirring bar were added nonafluorobutyl thianthrenium salt 2 (734 mg, 1.00 mmol, 1.00 equiv.) and anhydrous MeCN (7.2 mL, 0.14 M). The flask was capped with a rubber septum, and subsequently [1.1.1]propellane solution in Et₂O (c=1.0 M, 1.2 mL, 1.2 equiv.) was added dropwise via syringe to the reaction over 10 min while stirring. Subsequently, the reaction flask was placed in front of two Kessil PR160-390 nm LEDs. The mixture was irradiated for 4 h while maintaining the temperature at approximately 35° C. through cooling with a fan. After 4 h, the reaction flask was removed from the two Kessil PR160-390 nm LEDs. The mixture was concentrated under reduced pressure, diluted with DCM (20 mL). The DCM solution was poured into a separation funnel and washed with aqueous NaBF₄ solution (3×ca. 20 mL, 10% w/w). All organic phases were combined, dried over MgSO₄ (10 g), filtered, and the solvent evaporated under reduced pressure. The crude material was purified by chromatography on silica gel eluting first with 100% EtOAc and later DCM/MeOH (100/0-98/2 (v/v)) to afford the title compound 4 as a brown solid (427 mg, 726 μmol, 73%).

$${R}_{f} = 0.53 ( DCM / MeOH , 10 : 1 ( v / v ) ) .$$

Melting point: 112-113° C.

HRMS-ESI (m/z) calculated for C₂₁H₁₄S₂F₉⁺ [M-BF₄]⁺, 501.0390; found, 501.0387; deviation: −0.6 ppm.

### Thianthrenium tetrafluoroborate (TT^{⋅+} BF₄⁻)

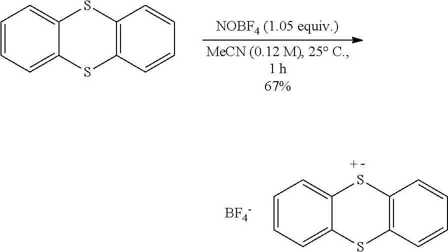

Thianthrenium tetrafluoroborate was synthesized according to a reported procedure⁵. In a nitrogen-filled glove box, thianthrene (2.00 g, 9.25 mmol, 1.00 equiv) was added to nitrosonium tetrafluoroborate (1.13 g, 9.71 mmol, 1.05 equiv) in acetonitrile (80 mL, c=0.12 M) to produce a dark purple solution. The glove box was purged while the reaction mixture was stirred for 1 h at 25° C., at which point, diethyl ether (250 mL) was added to the stirred reaction mixture. The precipitate was collected by filtration and washed with diethyl ether until the filtrate was colorless (5×10 mL). The filter cake was transferred to a 20 mL borosilicate vial and put under vacuum for 5 h, yielding the title compound as a free-flowing black-purple solid (1.88 g, 6.20 mmol, 67% yield).

Melting point: 178-179° C. The melting process is accompanied by decomposition.

### Determination of Purity:

A precise amount (approx. 150 mg) of the thianthrenium tetrafluoroborate was dissolved in anhydrous DCM (30 mL) and MeCN (5 mL) under N₂ atmosphere. KI (500 mg, 3.00 mmol) was added, and the mixture was stirred until the deep purple color of TT^{⋅+} had been replaced with the dark red brown color of I₂. The liberated iodine was titrated with standard sodium thiosulfate. The procedure was repeated twice, and assays were 99.5% and 98.5% of TT^{⋅+} BF₄⁻.

### Cyanobicyclo[1.1.1]pentyl thianthrenium salt (5)

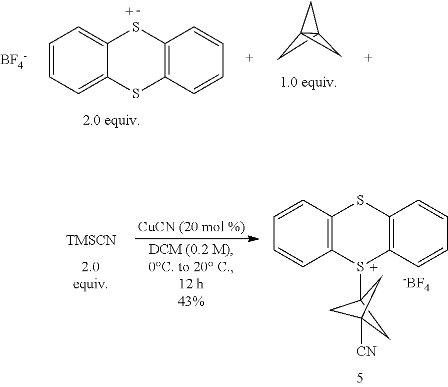

Under nitrogen atmosphere, to a 4 mL borosilicate vial equipped with a magnetic stir bar were added thianthrenium tetrafluoroborate (TT^{⋅+} BF₄⁻) (120 mg, 0.200 mmol, 2.00 equiv.), CuCN (7.2 mg, 80 μmoL, 40 mol %), and anhydrous DCM (1.0 mL, c=0.2 M). The vial was sealed with a septum-cap. After cooling to 0° C., TMSCN (50 μL, 40 mg, 0.40 mmol, 2.0 equiv.) was added in one portion. The mixture was stirred for 10 min at 0° C. Subsequently, [1.1.1]propellane solution in Et₂O (c=0.90 M) (0.22 mL, 0.20 mmol, 1.0 equiv.) was added to the mixture. Then, the mixture was stirred at 0° C. for 3 h, followed by stirring at 20° C. for 9 h. The mixture was diluted with MeCN (3 mL), and washed with aqueous NaBF₄ solution (1×3 mL, 10% w/w). The resulting mixture was poured into a separation funnel, vigorously shaken, and the layers were separated. The aqueous layer was extracted with DCM (2×5 mL). The combined organic phase was dried over Na₂SO₄, filtered, and the solvent was removed under reduced pressure. The residue was purified by chromatography on silica gel eluting with DCM/MeOH (1/0-30/1 (v/v)) to afford the title compound 5 as a pale brown solid (43.4 mg, 0.110 mmol, 55%).

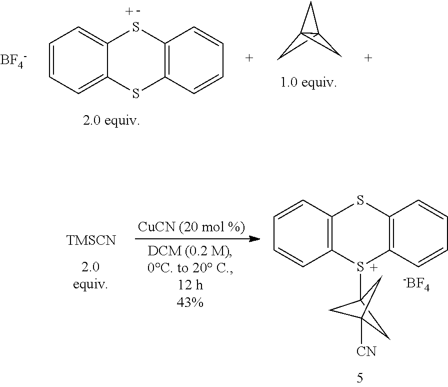

Under nitrogen atmosphere, to a 50 mL round-bottom flask equipped with a magnetic stir bar were added thianthrenium tetrafluoroborate (TT^{⋅+} BF₄⁻) (3.18 g, 10.5 mmol, 1.98 equiv.), CuCN (95 mg, 1.1 mmoL, 20 mol %), and anhydrous DCM (26 mL, c=0.20 M). The flask was sealed with a septum-cap. After cooling to 0° C., TMSCN (1.33 mL, 1.05 g, 10.6 mmol, 2.00 equiv.) was added in one portion. The mixture was stirred for 10 min at 0° C. Subsequently, [1.1.1]propellane solution in Et₂O (c=0.71 M) (7.5 mL, 5.3 mmol, 1.0 equiv.) was added to the mixture. Then, the mixture was stirred at 0° C. for 3 h, followed by stirring at 20° C. for 9 h. The mixture was diluted with DCM (20 mL) and MeCN (20 mL), and washed with aqueous NaBF₄ solution (1×20 mL, 10% w/w). The resulting mixture was poured into a separation funnel, vigorously shaken, and the layers were separated. The aqueous layer was extracted with DCM (2×50 mL). The combined organic phase was dried over Na₂SO₄, filtered, and the solvent was removed under reduced pressure. The residue was purified by fast chromatography on silica gel eluting with DCM/MeOH (1/0-30/1 (v/v)) to afford the title compound 5 as a pale brown solid (890 mg, 2.25 mmol, 43%).

$${R}_{f} = 0.47 ( DCM / MeOH , 10 : 1 ( v / v ) ) .$$

Melting point: 147-148° C.

HRMS-ESI (m/z) calculated for C₁₈H₁₄NS₂⁺ [M-BF₄]⁺, 308.0560; found, 308.0562; deviation: +0.8 ppm.

### 4-Toluenesulfonylbicyclo[1.1.1] pentyl phenoxathiinium salt (8)

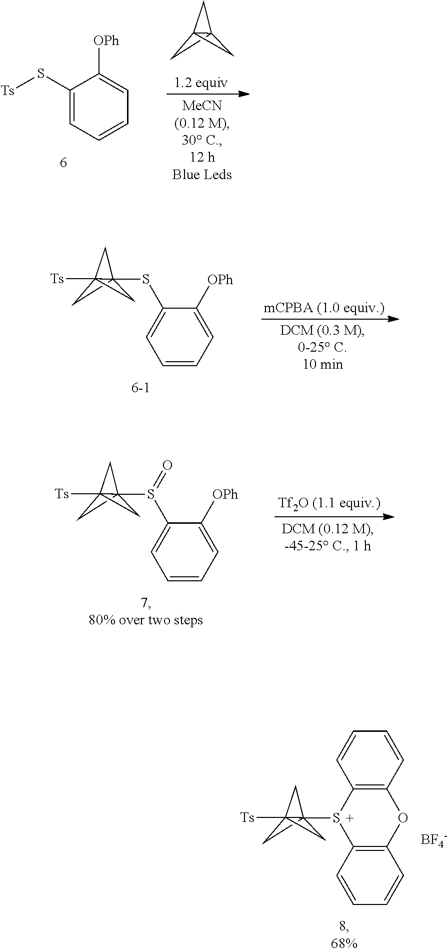

Under nitrogen atmosphere, to a 25 mL round-bottom flask equipped with a magnetic stir bar were added thiosulfonate 6 (1.5 g, 4.2 mmol, 1.0 equiv.), [1.1.1]propellane solution in Et₂O (c=0.82 M, 5.0 mmol, 6.2 mL, 1.2 equiv.), and CH₃CN (35 mL, c=0.12 M). Subsequently, the reaction flask was placed 5 cm away from two blue LEDs (Kessil A160WE Tuna Blue (460 nm), LED lighting, 40 W). The mixture was irradiated for 12 h while maintaining the temperature at approximately 30° C. through cooling with a fan. The mixture was concentrated under reduced pressure to afford the crude compound 6-1, which was directly used for the next step without further purification.

To a solution of 6-1 in DCM (14 mL, c=0.30 M) at 0° C. was added 3-chloroperoxybenzoic acid (≤77%) (0.98 g, 4.4 mmol, 1.0 equiv.). The mixture was stirred for 10 min at 25° C. and then diluted with DCM (10 mL). The suspension was filtered, and the filtrate was washed with saturated Na₂S₂O₃ solution (2×5 mL) and 1 M NaOH solution (2×5 mL). The combined organic phase was dried over Na₂SO₄, the resulting suspension was filtered, and the solvent was removed under reduced pressure. The residue was purified by chromatography on silica gel eluting with EtOAc/pentane (1/10-1/2 (v/v)) to afford the 7 as a colorless solid (1.48 g, 3.37 mmol, 80%).

$${R}_{f} = 0.37 ( pentane / EtOAc , 1 : 1 ( v / v ) ) .$$

Melting point: 148-149° C.

HRMS-ESI (m/z) calculated for C₂₄H₂₂NaO₄S₂⁺ [M+Na]⁺, 461.0849; found, 461.0852; deviation: +0.7 ppm.

Under an ambient atmosphere, to a 50 mL round-bottom flask equipped with a magnetic stir bar were added 7 (1.26 g, 2.87 mmol, 1.00 equiv.) and DCM (24 mL, c=0.12 M). The suspension was cooled to −45° C. (dry ice/isopropanol bath), and subsequently trifluoromethanesulfonic anhydride (0.53 mL, 0.93 g, 3.2 mmol, 1.1 equiv.) were added over 1 min. The mixture was stirred at −45° C. for 10 min, and subsequently 50 min at 25° C. The mixture was diluted with DCM (20 mL) and NaBF₄ solution (15 mL, 10% w/w). The mixture was poured into a separation funnel, vigorously shaken, and the layers were separated. The organic phase was washed with aqueous NaBF₄ solution (2×15 mL, 10% w/w). The aqueous layer was extracted with DCM (1×50 mL). The combined organic phase was dried over Na₂SO₄, filtered, and the solvent was removed under reduced pressure. The residue was purified by fast chromatography on silica gel eluting with DCM/MeOH (1/0-30/1 (v/v)) to afford 8 as a colorless solid (992 mg, 1.95 mmol, 68%).

$${R}_{f} = 0.45 ( DCM / MeOH , 10 : 1 ( v / v ) ) .$$

Melting point: 137-138° C.

HRMS-ESI (m/z) calculated for C₂₄H₂₁O₃S₂⁺ [M-BF₄]⁺, 421.0927; found, 421.0927; deviation: +0.0 ppm.

General Procedure for Cu-Catalyzed C—O Coupling of 3-5 with Phenols

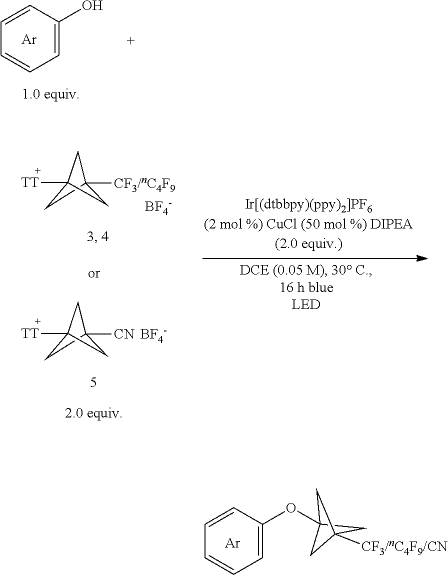

Under nitrogen atmosphere, to a 4 mL borosilicate vial equipped with a magnetic stir bar were added phenol (0.150 mmol, 1.00 equiv.), 3-5 (0.300 mmol, 2.00 equiv.), Ir[(dtbbpy)(ppy)₂]PF₆ (3 mg, 3 μmoL, 2 mol %), CuCl (7.4 mg, 75 μmoL, 50 mol %), anhydrous DCE (3.0 mL, c=50 mM), and DIPEA (52 μL, 39 mg, 0.30 mmol, 2.0 equiv.). The vial was sealed with a septum-cap. Then, the mixture was stirred for 10 min at 25° C., and placed 5 cm away from two blue LEDs (Kessil A160WE Tuna Blue (460 nm), LED lighting, 40 W). The mixture was irradiated for 16 h while maintaining the temperature at approximately 30° C. through cooling with a fan. After irradiation, the mixture was concentrated under reduced pressure. The residue was purified by flash column chromatography on silica gel to afford the desired product.

General Procedure A for Cu-Catalyzed C—N Coupling of 3-5 and 8 with N-Nucleophiles

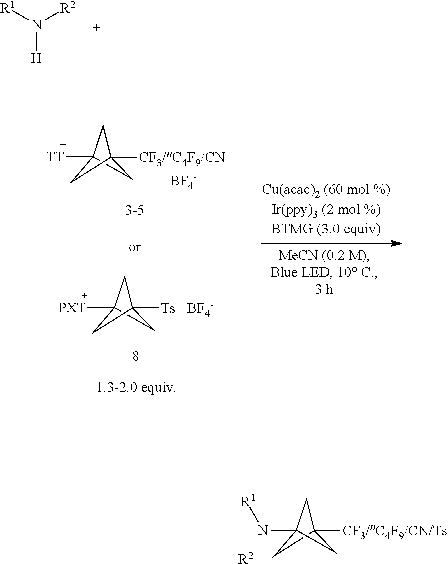

Under air, a 4 mL borosilicate vial equipped with a magnetic stir bar was charged with the N-nucleophile (0.300 mmol, 1.00 equiv.), 3-5 or 8 (1.30-2.00 equiv.), Ir(ppy)₃ (4 mg, 6 μmol, 2 mol %), and Cu(acac)₂ (47 mg, 0.18 mmol, 60 mol %). The vial was sealed with a septum-cap, evacuated, and flushed with argon three times using Schlenk technique. Under positive pressure of argon of 0.1 bars, 2-tert-butyl-1,1,3,3-tetramethylguanidine (BTMG) (0.18 mL, 0.15 g, 0.90 mmol, 3.0 equiv.) was added, followed by anhydrous MeCN (1.5 mL, c=0.20 M). The mixture was irradiated for 3 h at 10° C. using a photoreactor equipped with a blue LED module (KT-Elektronik, “100 W Power LED blau 450 nm Aquarium”, 450 nm, 60 W) (FIG. S8), cooled with two Peltier-elements (TEC1-12706). After irradiation, the mixture was concentrated under reduced pressure. The residue was purified by flash column chromatography on silica gel to afford the desired product.

General Procedure B for Cu-Catalyzed C—N Coupling of 3-5 and 8 with N-Nucleophiles

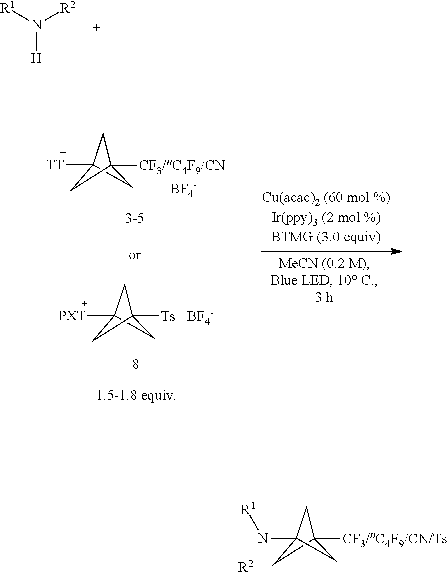

Under air, a 4 mL borosilicate vial equipped with a magnetic stir bar was charged with 3-5 or 8 (0.250 mmol, 1.00 equiv.), the N-nucleophile (1.50-1.80 equiv.), Ir(ppy)₃ (3.3 mg, 5.0 μmol, 2 mol %), and Cu(acac)₂ (39.3 mg, 0.150 mmol, 60 mol %). The vial was sealed with a septum-cap, evacuated, and flushed with argon three times using Schlenk technique. Under positive pressure of argon of 0.1 bars, 2-tert-butyl-1,1,3,3-tetramethylguanidine (BTMG) (0.15 mL, 0.13 g, 0.75 mmol, 3.0 equiv.) was added, followed by anhydrous MeCN (1.3 mL, c=0.20 M). The mixture was irradiated for 3 h at 10° C. using a photoreactor equipped with a blue LED module (KT-Elektronik, “100 W Power LED blau 450 nm Aquarium”, 450 nm, 60 W) (FIG. S8), cooled with two Peltier-elements (TEC1-12706). After irradiation, the mixture was concentrated under reduced pressure. The residue was purified by flash column chromatography on silica gel to afford the desired product.

General Procedure for Ni-Catalyzed Reductive C—C Coupling of 3-5 with Aryl Bromides

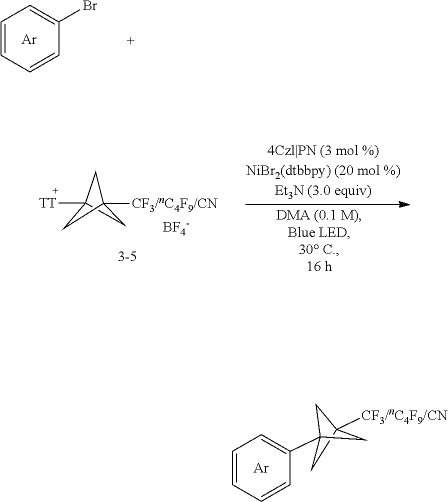

Under nitrogen atmosphere, to a 4 mL borosilicate vial equipped with a magnetic stir bar were added aryl bromide (0.200 mmol, 1.00 equiv.), 3-5 (0.300 mmol, 1.50 equiv.), 4CzIPN (5 mg, 6 μmoL, 3 mol %), Ni(dtbbpy)Br₂ (19.4 mg, 40.0 μmoL, 20.0 mol %), anhydrous DMA (2.0 mL, c=0.10 M), and Et₃N (83 μL, 61 mg, 0.60 mmol, 3.0 equiv.). The vial was sealed with a septum-cap. Then, the mixture was stirred for 10 min at 25° C., and placed 5 cm away from two blue LEDs (Kessil A160WE Tuna Blue (460 nm), LED lighting, 40 W). The mixture was irradiated for 16 h while maintaining the temperature at approximately 30° C. through cooling with a fan. After irradiation, EtOAc (6 mL) was added to the mixture, and washed with brine (2×3 mL). The organic phase was dried over Na₂SO₄, filtered, and the solvent was removed under reduced pressure. The residue was purified by flash column chromatography on silica gel to afford the desired product.

### Bicyclo[1.1.1]pentylether 13

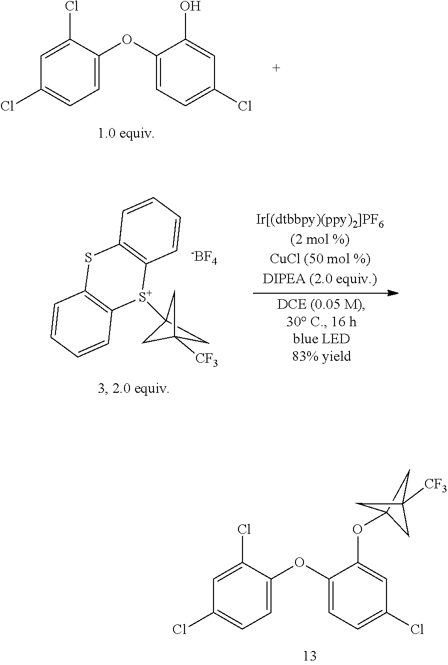

Under nitrogen atmosphere, to a 4 mL borosilicate vial equipped with a magnetic stir bar were triclosan (45.2 mg, 0.150 mmol, 1.00 equiv.), 3 (130 mg, 0.300 mmol, 2.00 equiv.), Ir[(dtbbpy)(ppy)₂]PF₆ (3 mg, 3 μmoL, 2 mol %), CuCl (7.4 mg, 75 μmoL, 50 mol %), anhydrous DCE (3.0 mL, c=50 mM), and DIPEA (52 μL, 39 mg, 0.30 mmol, 2.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, the mixture was concentrated under reduced pressure. The residue was purified by flash column chromatography on silica gel eluting with pentane to afford the title compound 13 as a colorless oil (52.9 mg, 0.125 mmol, 83%).

$${R}_{f} = 0.5 ( pentanes / EtOAc , 95 : 5 ( v / v ) ) .$$

HRMS-EI (m/z) calc'd for C₁₈H₁₂O₂F₃Cl₃⁺ [M]⁺, 421.9855; found, 421.9849; deviation: −1.2 ppm.

### Bicyclo[1.1.1]pentylether 14

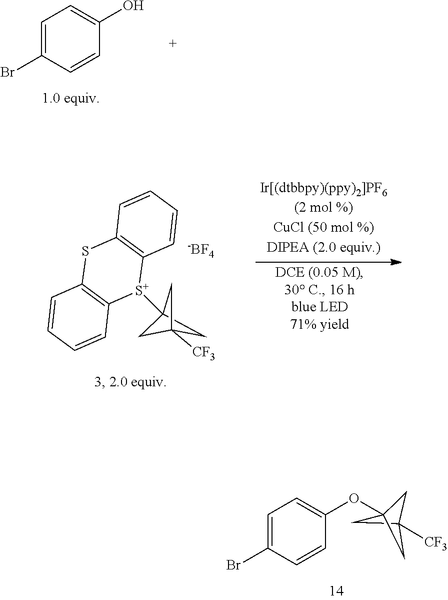

Under nitrogen atmosphere, to a 4 mL borosilicate vial equipped with a magnetic stir bar were added 4-bromo phenol (31.1 mg, 0.180 mmol, 1.00 equiv.), 3 (160 mg, 0.360 mmol, 2.00 equiv.), Ir[(dtbbpy)(ppy)₂]PF₆ (3 mg, 3 μmoL, 2 mol %), CuCl (8.9 mg, 90 μmoL, 50 mol %), anhydrous DCE (3.0 mL, c=60 mM), and DIPEA (63 μL, 47 mg, 0.36 mmol, 2.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, the mixture was concentrated under reduced pressure. The residue was purified by flash column chromatography on silica gel eluting with EtOAc/pentane (0:100-1:50 (v/v)) to afford the title compound 14 as a yellow oil (39.2 mg, 0.128 mmol, 71%).

$${R}_{f} = 0.5 ( pentane / EtOAc , 20 : 1 ( v / v ) ) .$$

HRMS-CI (m/z) calc'd for C₁₂H₁₁OF₃Br⁺ [M+H]⁺, 306.9939; found, 306.9940; deviation: +0.4 ppm.

### Bicyclo[1.1.1]pentylether 18

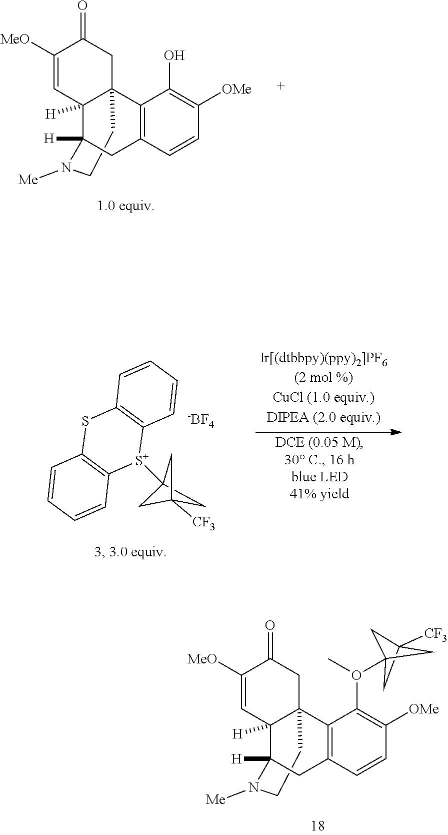

Under nitrogen atmosphere, to a 4 mL borosilicate vial equipped with a magnetic stir bar were added sinomenine (32.9 mg, 0.100 mmol, 1.00 equiv.), 3 (130 mg, 0.300 mmol, 3.00 equiv.), Ir[(dtbbpy)(ppy)₂]PF₆ (2 mg, 2 μmoL, 2 mol %), CuCl (9.9 mg, 0.10 mmoL, 1.0 equiv), anhydrous DCE (2.0 mL, c=50 mM), and DIPEA (35 μL, 26 mg, 0.20 mmol, 2.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, the mixture was concentrated under reduced pressure. The residue was purified by flash column chromatography on silica gel eluting with MeOH/DCM (0:100-1:20 (v/v)) to afford the title compound 18 as a yellow oil (19.0 mg, 41.0 μmol, 41%).

$${R}_{f} = 0.3 ( MeOH / DCM , 1 : 10 ( v / v ) ) .$$

$${[ α ]}_{D}^{25} = - 52.9 ( c = 0.14 , {CHCl}_{3} ) .$$

HRMS-ESI (m/z) calc'd for C₂₅H₂₉NO₄F₃⁺ [M+H]⁺, 464.2043; found, 464.2046; deviation: +0.6 ppm.

### Bicyclo[1.1.1]pentylether 32

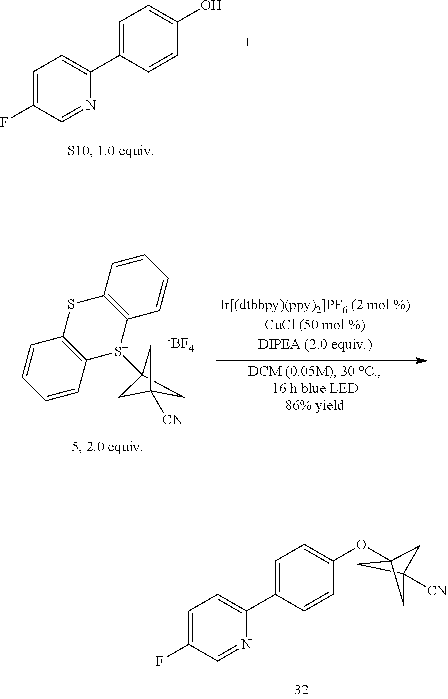

Under nitrogen atmosphere, to a 4 mL borosilicate vial equipped with a magnetic stir bar were added S10 (18.9 mg, 0.100 mmol, 1.00 equiv.), 5 (79.0 mg, 0.200 mmol, 2.00 equiv.), Ir[(dtbbpy)(ppy)₂]PF₆ (2 mg, 2 μmoL, 2 mol %), CuCl (4.9 mg, 50 μmoL, 50 mol %), anhydrous DCM (2.0 mL, c=0.05 M), and DIPEA (35 μL, 26 mg, 0.20 mmol, 2.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, the mixture was concentrated under reduced pressure. The residue was purified by flash column chromatography on silica gel eluting with EtOAc/pentane (0:100-1:8 (v/v)) to afford the title compound 32 as a colorless oil (24.0 mg, 86.0 μmol, 86%).

$${R}_{f} = 0.5 ( pentane / EtOAc , 3 : 1 ( v / v ) ) .$$

HRMS-EI (m/z) calc'd for C₁₇H₁₃N₂OF⁺ [M]⁺, 280.1007; found, 280.1006; deviation: +0.1 ppm.

### Bicyclo[1.1.1]pentylamine 49

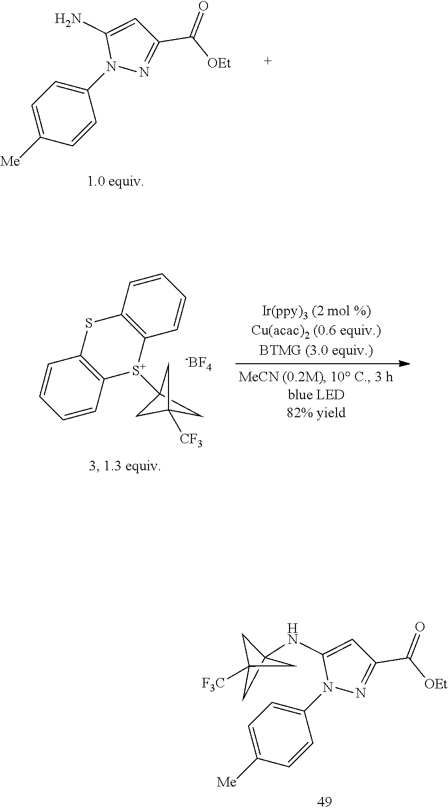

A 4 mL borosilicate vial equipped with a magnetic stir bar was charged with ethyl-5-amino-1-(4-methylphenyl)-1H-pyrazol-4-carboxylate (40.0 mg, 0.163 mmol, 1.00 equiv.), 3 (92.9 mg, 0.212 mmol, 1.30 equiv.), Ir(ppy)₃ (2.1 mg, 3.3 μmol, 2.0 mol %), and Cu(acac)₂ (25.6 mg, 98.0 μmol, 0.600 equiv.). The vial was sealed with a septum-cap, evacuated, and flushed with argon three times using Schlenk technique. Under positive pressure of argon, anhydrous MeCN (1.0 mL, c=0.20 M) was added, followed by 2-tert-butyl-1,1,3,3-tetramethylguanidine (0.10 mL, 84 mg, 0.49 mmol, 3.0 equiv.) while stirring. The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with hexanes/EtOAc (1/0-1/4 (v/v)) to afford the title compound 49 as a light yellow oil (51.0 mg, 0.134 mmol, 82%). R_{f}=0.80 (EtOAc/hexanes, 1:1 (v/v)).

HRMS-ESI (m/z) calculated for C₁₉H₂₁N₃F₃O₂⁺ [M+H]⁺, 380.1579; found, 380.1580; deviation: +0.2 ppm.

### Bicyclo[1.1.1]pentylamine 50

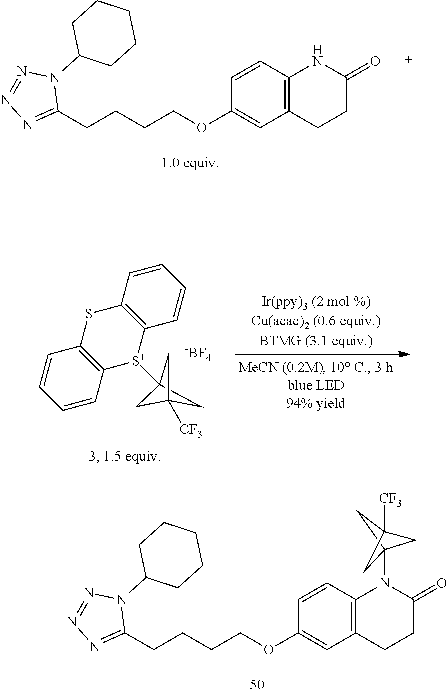

A 4 mL borosilicate vial equipped with a magnetic stir bar was charged with cilostazol (0.11 g, 0.30 mmol, 1.0 equiv.), 3 (0.20 g, 4.5 mmol, 1.5 equiv.), Ir(ppy)₃ (4 mg, 6 μmol, 2 mol %), and Cu(acac)₂ (47 mg, 0.18 mmol, 0.60 equiv.). The vial was sealed with a septum-cap, evacuated, and flushed with argon three times using Schlenk technique. Under positive pressure of argon, anhydrous MeCN (1.5 mL, c=0.20 M) was added, followed by 2-tert-butyl-1,1,3,3-tetramethylguanidine (0.19 mL, 0.16 g, 0.94 mmol, 3.1 equiv.) while stirring. The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with EtOAc/hexanes (3:10-9:20 (v/v)) to afford the title compound 50 as a brown solid (142 mg, 0.281 mmol, 94%).

$${R}_{f} = 0.3 ( EtOAc / hexanes , 3 : 2 ( v / v ) ) .$$

Melting point: 109-110° C.

HRMS-ESI (m/z) calculated for C₂₆H₃₂N₅O₂F₃Na⁺ [M+Na]⁺, 526.2407; found, 526.2400; deviation: −1.2 ppm.

### Bicyclo[1.1.1]pentylamine 59

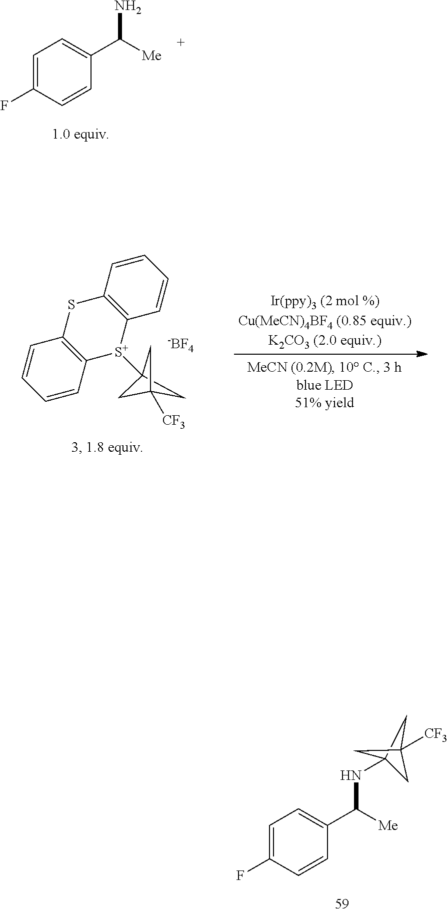

A 4 mL borosilicate vial equipped with a magnetic stir bar was charged with (S)-4-fluoro-α-methylbenzylamine (40.0 μL, 0.300 mmol, 1.00 equiv.), 3 (236 mg, 0.538 mmol, 1.80 equiv.), Ir(ppy)₃ (6.0 mg, 9.2 μmol, 2.0 mol %), K₂CO₃ (83 mg, 0.60 mol, 2.0 equiv., and Cu(MeCN)₄BF₄ (80.5 mg, 0.256 mmol, 0.853 equiv.). The vial was sealed with a septum-cap, evacuated, and flushed with argon three times using Schlenk technique. Under positive pressure of argon, anhydrous MeCN (1.5 mL, c=0.20 M) was added while stirring. The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with EtOAc/hexanes (5:100-3:25 (v/v)) to afford the title compound 59 as a yellow oil (42.0 mg, 0.154 mmol, 51%).

$${R}_{f} = 0.3 ( EtOAc / hexanes , 1 : 5 ( v / v ) ) .$$

$${[ α ]}_{D}^{25} = - 21. ( c = 1.17 , {CHCl}_{3} ) .$$

HRMS-EI (m/z) calculated for C₁₄H₁₅NF₄⁺ [M]⁺, 273.1135; found, 273.1135; deviation: +0.2 ppm.

### Bicyclo[1.1.1]pentylamine 60

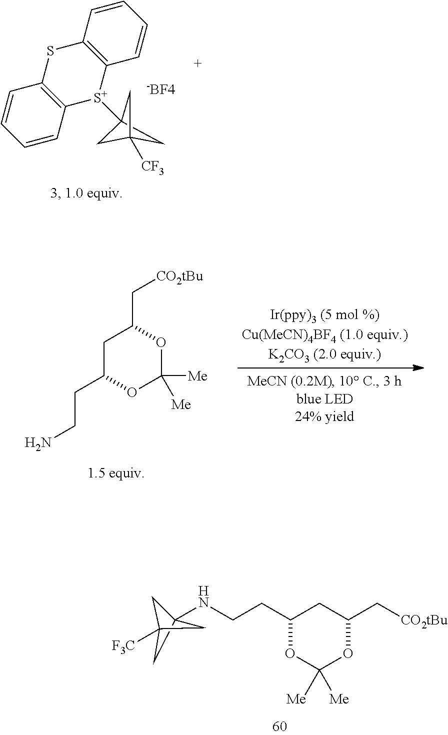

A 4 mL borosilicate vial equipped with a magnetic stir bar was charged with 3 (131 mg, 0.300 mmol, 1.00 equiv.), tert-butyl 2-(4R-cis)-6-aminoethyl-2,2-dimethyl-1,3-dioxane-4-acetate (123 mg, 0.450 mmol, 1.50 equiv.), Ir(ppy)₃ (10 mg, 15 μmol, 5.0 mol %), K₂CO₃ (83 mg, 0.60 mol, 2.0 equiv), and Cu(MeCN)₄BF₄ (94 mg, 0.30 mmol, 1.0 equiv.). The vial was sealed with a septum-cap, evacuated, and flushed with argon three times using Schlenk technique. Under positive pressure of argon, anhydrous MeCN (1.5 mL, c=0.20 M) was added while stirring. The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with EtOAc/hexanes (10:100-3:25 (v/v)) to afford the title compound 60 as a yellow oil (29 mg, 0.07 mmol, 24%).

$${R}_{f} = 0.4 ( EtOAc / hexanes , 1 : 1 ( v / v ) ) .$$

$${[ α ]}_{D}^{25} = + 9.4 ( c = 0.68 , {CHCl}_{3} ) .$$

HRMS-ESI (m/z) calculated for C₂₀H₃₃NF₃O₄⁺ [M+H]⁺, 408.2358; found, 408.2356; deviation: −0.3 ppm.

### Bicyclo[1.1.1]pentylmetaxalone 61

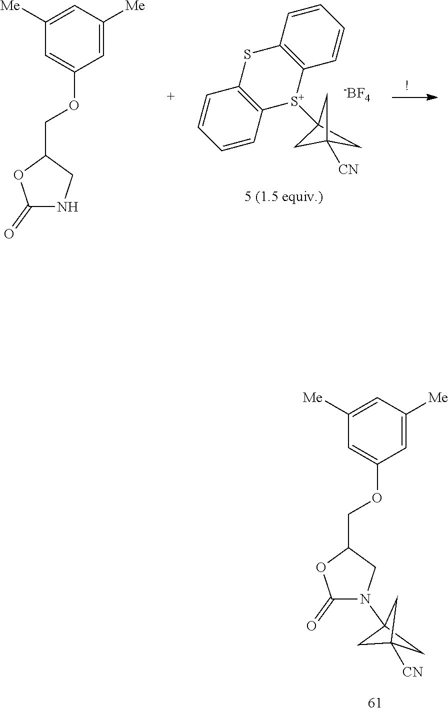

A 4 mL borosilicate vial equipped with a magnetic stir bar was charged with metaxalone (22.1 mg, 0.100 mmol, 1.00 equiv.), 2 (59.3 mg, 0.150 mmol, 1.50 equiv.), Ir(ppy)₃ (0.7 mg, 1 μmol, 1 mol %), and Cu(acac)₂ (15.5 mg, 60.0 μmol, 0.60 equiv.). The vial was sealed with a septum-cap, evacuated, and flushed with argon three times using Schlenk technique. Under positive pressure of argon, 2-tert-butyl-1,1,3,3-tetramethylguanidine (61 μL, 51 mg, 0.30 mmol, 3.0 equiv.) was added, followed by anhydrous MeCN (0.5 mL, c=0.2 M). The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with EtOAc/pentane (1/5-2/1 (v/v)) to afford the title compound xx as a pale yellow oil (27.8 mg, 89.0 μmol, 89%).

$${R}_{f} = 0.57 ( pentane / EtOAc , 1 : 1 ( v / v ) ) .$$

HRMS-EI (m/z) calculated for C₁₈H₂₀N₂O₃⁺ [M]⁺, 312.1471; found, 312.1468; deviation: −0.7 ppm.

### Bicyclo[1.1.1]pentylazaindole 62

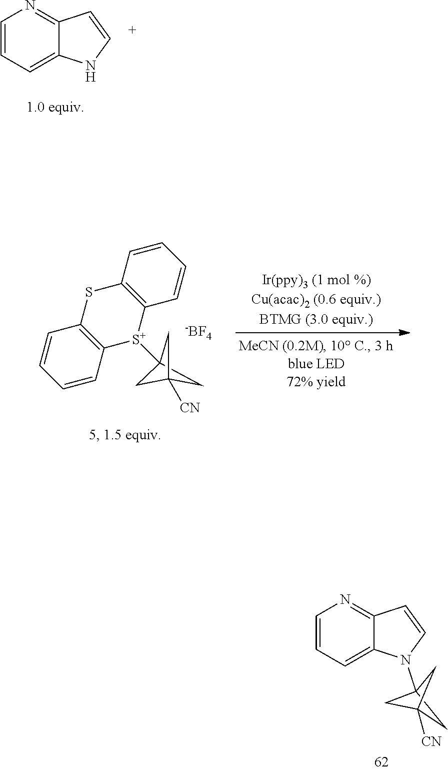

A 4 mL borosilicate vial equipped with a magnetic stir bar was charged with 1H-pyrrolo[3,2-b]pyridine (11.8 mg, 0.100 mmol, 1.00 equiv.), 5 (59.3 mg, 0.150 mmol, 1.50 equiv.), Ir(ppy)₃ (0.7 mg, 1 μmol, 1 mol %), and Cu(acac)₂ (15.5 mg, 60.0 μmol, 0.60 equiv.). The vial was sealed with a septum-cap, evacuated, and flushed with argon three times using Schlenk technique. Under positive pressure of argon, 2-tert-butyl-1,1,3,3-tetramethylguanidine (61 μL, 51 mg, 0.30 mmol, 3.0 equiv.) was added, followed by anhydrous MeCN (0.5 mL, c=0.2 M). The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with EtOAc/pentane (0/100-1/1 (v/v)) to afford the title compound 62 as a pale yellow solid (15.0 mg, 71.7 μmol, 72%).

$${R}_{f} = 0.17 ( pentane / EtOAc , 1 : 1 ( v / v ) ) .$$

Melting point: 173-174° C.

HRMS-EI (m/z) calculated for C₁₃H₁₁N₃⁺ [M]⁺, 209.0947; found, 209.0947; deviation: −0.2 ppm.

### Bicyclo[1.1.1]pentylmetaxalone 63

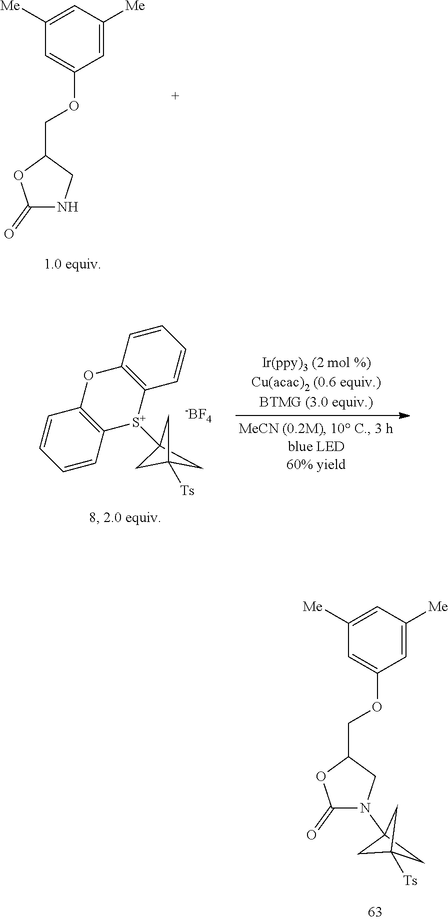

A 4 mL borosilicate vial equipped with a magnetic stir bar was charged with metaxalone (22.1 mg, 0.100 mmol, 1.00 equiv.), 8 (102 mg, 0.200 mmol, 1.50 equiv.), Ir(ppy)₃ (1.4 mg, 2 μmol, 2 mol %), and Cu(acac)₂ (15.5 mg, 60.0 μmol, 0.60 equiv.). The vial was sealed with a septum-cap, evacuated, and flushed with argon three times using Schlenk technique. Under positive pressure of argon, anhydrous MeCN (0.5 mL, c=0.2 M) was added, followed by 2-tert-butyl-1,1,3,3-tetramethylguanidine (61 μL, 51 mg, 0.30 mmol, 3.0 equiv.) while stirring. The mixture was irradiated for 3 h at 10° C. using a photoreactor equipped with a blue LED module (KT-Elektronik, “100 W Power LED blau 450 nm Aquarium”, 450 nm, 60 W), cooled with two Peltier-elements (TEC1-12706). Then, the mixture was concentrated to dryness. The residue was purified by chromatography on silica gel eluting with EtOAc/pentane (1/5-2/1 (v/v)) to afford the title compound 63 as a pale yellow oil (26.5 mg, 60.0 μmol, 60%).

$${R}_{f} = 0.37 ( pentane / EtOAc , 1 : 1 ( v / v ) ) .$$

HRMS-ESI (m/z) calculated for C₂₄H₂₇NNaO₅S⁺ [M+Na]⁺, 464.1505; found, 464.1502; deviation: −0.6 ppm.

### Bicyclo[1.1.1]pentylarene 77

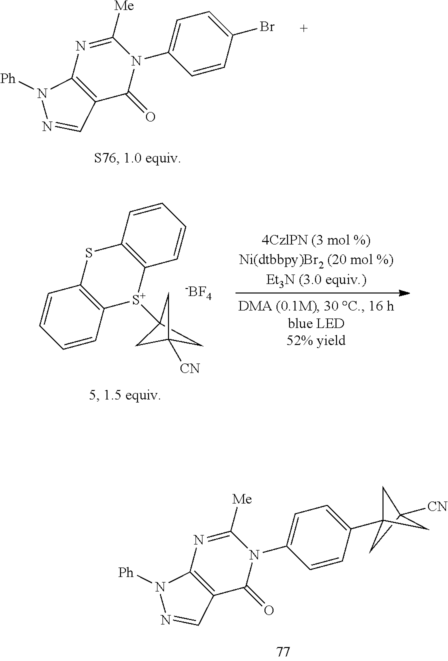

Under nitrogen atmosphere, to a 4 mL borosilicate vial equipped with a magnetic stir bar were added S76 (38.1 mg, 0.100 mmol, 1.00 equiv.), 5 (59.3 mg, 0.150 mmol, 1.50 equiv.), 4CzIPN (2.5 mg, 3.0 μmoL, 3 mol %), Ni(dtbbpy)Br₂ (9.7 mg, 20 μmoL, 20 mol %), anhydrous DMA (1.0 mL, c=0.10 M), and Et₃N (42 μL, 30 mg, 0.30 mmol, 3.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, EtOAc (6 mL) was added to the mixture, and washed with brine (2×3 mL). The organic phase was dried over Na₂SO₄, filtered, and the solvent was removed under reduced pressure. The residue was purified by flash column chromatography on silica gel eluting with EtOAc/pentane (0:100-1:5 (v/v)) to afford the title compound 77 as a colorless solid (20.4 mg, 51.8 μmol, 52%).

$${R}_{f} = 0.45 ( pentane / EtOAc , 2 : 1 ( v / v ) ) .$$

Melting point: 286-287° C.

HRMS-ESI (m/z) calc'd for C₂₄H₁₉N₅ONa⁺ [M+Na]⁺, 416.1480; found, 416.1482; deviation: +0.4 ppm.

### Bicyclo[1.1.1]pentylarene 82

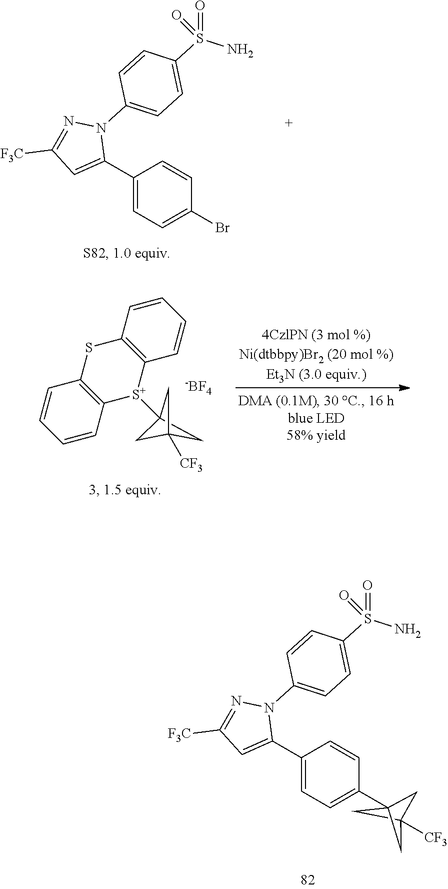

Under nitrogen atmosphere, to a 4 mL borosilicate vial equipped with a magnetic stir bar were added S82 (89.2 mg, 0.200 mmol, 1.00 equiv.), 3 (130 mg, 0.300 mmol, 1.50 equiv.), 4CzIPN (5 mg, 6 μmoL, 3 mol %), Ni(dtbbpy)Br₂ (19.4 mg, 40.0 μmoL, 20.0 mol %), anhydrous DMA (2.0 mL, c=0.10 M), and Et₃N (83 μL, 61 mg, 0.60 mmol, 3.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, EtOAc (6 mL) was added to the mixture, and washed with brine (2×3 mL). The organic phase was dried over Na₂SO₄, filtered, and the solvent was removed under reduced pressure. The residue was purified by flash column chromatography on silica gel eluting with EtOAc/pentane (0:100-1:6 (v/v)) to afford the title compound 82 as a colorless solid (58.2 mg, 0.116 mmol, 58%).

$${R}_{f} = 0.54 ( pentane / EtOAc , 3 : 1 ( v / v ) ) .$$

Melting point: 120-121° C.

HRMS-ESI (m/z) calc'd for C₂₂H₁₆N₃O₂S₁F₆ [M−H]⁻, 500.0877; found, 500.0873; deviation: 0.7 ppm.

### Bicyclo[1.1.1]pentylarene 87

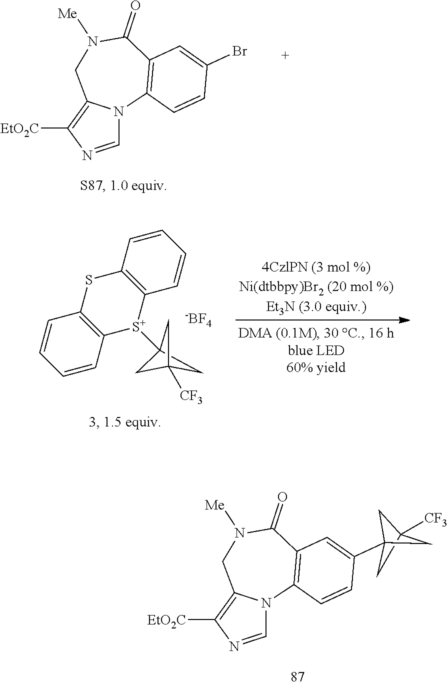

Under nitrogen atmosphere, to a 4 mL borosilicate vial equipped with a magnetic stir bar were added S87 (72.6 mg, 0.200 mmol, 1.00 equiv.), 3 (130 mg, 0.300 mmol, 1.50 equiv.), 4CzIPN (5 mg, 6 μmoL, 3 mol %), Ni(dtbbpy)Br₂ (19.4 mg, 40.0 μmoL, 20.0 mol %), anhydrous DMA (2.0 mL, c=0.10 M), and Et₃N (83 μL, 61 mg, 0.60 mmol, 3.0 equiv.). The vial was sealed with a septum-cap. Then, the mixture was stirred for 10 min at 25° C., and placed 5 cm away from two blue LEDs (Kessil A160WE Tuna Blue, LED lighting, 40 W). The mixture was irradiated for 16 h while maintaining the temperature at approximately 30° C. through cooling with a fan. After irradiation, EtOAc (6 mL) was added to the mixture, and washed with brine (2×3 mL). The organic phase was dried over Na₂SO₄, filtered, and the solvent was removed under reduced pressure. The residue was purified by flash column chromatography on silica gel eluting with DCM/MeOH (100:0-100:1 (v/v)) to afford the title compound 87 as a colorless solid (49.9 mg, 0.119 mmol, 60%).

$${R}_{f} = 0.3 ( DCM / MeOH , 25 : 1 ( v / v ) ) .$$

Melting point: 262-263° C.

HRMS-ESI (m/z) calc'd for C₂₁H₂₀F₃N₃O₃Na⁺ [M+Na]⁺, 442.1353; found, 442.1349; deviation: −0.9 ppm.

### Synthetic transformations of cyanobicyclo[1.1.1]pentylether 30

### Bicyclo[1.1.1]pentylalkyl amine 88

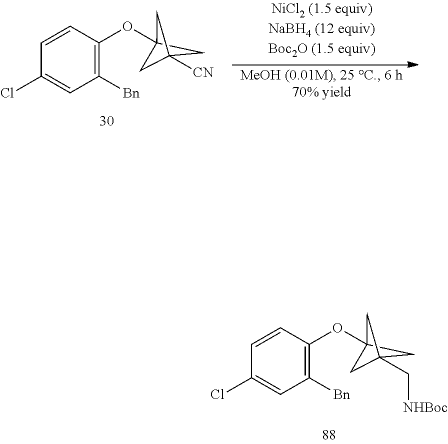 [SMILES: N#CC12CC(Oc3ccc(Cl)cc3Cc3ccccc3)(C1)C2 ; CC(C)(C)OC(=O)NCC12CC(Oc3ccc(Cl)cc3Cc3ccccc3)(C1)C2]

To a 25 mL round-bottom flask equipped with a magnetic stir bar were added 30 (30.9 mg, 0.100 mmol, 1.00 equiv), NiCl₂ (19.5 mg, 0.150 mmol, 1.50 equiv), Boc₂O (65.4 mg, 0.300 mmol, 3.00 equiv), and MeOH (10 mL, c=10 mM). The mixture was cooled to 0° C., and NaBH₄ (45.4 mg, 1.20 mmol, 12.0 equiv) was then added over 1 min. The mixture was stirred at 25° C. for 6 h. Then, saturated NH₄Cl solution (5 mL) was added to the mixture, which was then extracted with EtOAc (3×25 mL). The organic phase was dried over Na₂SO₄, filtered, and the solvent was removed under reduced pressure. The residue was purified by chromatography on silica gel eluting with EtOAc/pentane (1:50-1:10 (v/v)) to afford the title compound 88 as a colorless oil (28.9 mg, 70.0 μmol, 70%).

$${R}_{f} = 0.5 ( pentane / EtOAc , 5 : 1 ( v / v ) ) .$$

HRMS-ESI m/z calculated for C₂₄H₂₈ClNNaO₃⁺ [M+Na]⁺, 436.1650; found, 436.1650; deviation: −0.1 ppm.

### Bicyclo[1.1.1]pentylester 89

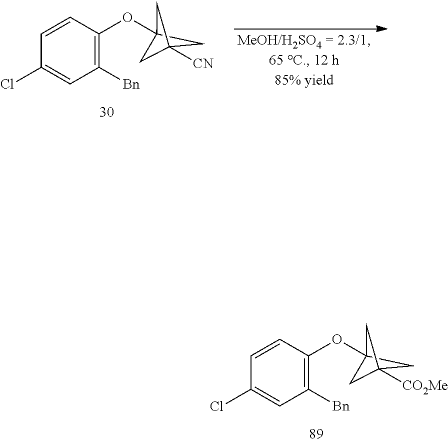 [SMILES: N#CC12CC(Oc3ccc(Cl)cc3Cc3ccccc3)(C1)C2 ; COC(=O)C12CC(Oc3ccc(Cl)cc3Cc3ccccc3)(C1)C2]

A 4 mL borosilicate vial equipped with a magnetic stir bar was charged with 30 (30.9 mg, 0.100 mmol, 1.00 equiv) and MeOH (800 μL, c=0.125 M). The mixture was cooled to 0° C., and concentrated H₂SO₄ (0.35 mL) was then added over 1 min. The mixture was stirred at 65° C. for 12 h. Cold water (5 mL) was added to the mixture, which was then extracted with EtOAc (3×15 mL). The organic phase was dried over Na₂SO₄, filtered, and the solvent was removed under reduced pressure. The residue was purified by chromatography on silica gel eluting with EtOAc/pentane (1:100-1:30 (v/v)) to afford the title compound 89 as a colorless oil (29.1 mg, 85.0 μmol, 85%).

$${R}_{f} = 0.31 ( pentane / EtOAc , 10 : 1 ( v / v ) ) .$$

HRMS-ESI (m/z) calculated for C₂₀H₁₉ClNaO₃⁺ [M+Na]⁺, 365.0916; found, 365.0915; deviation: −0.2 ppm.

### Bicyclo[1.1.1]pentyl carboxylic acid 90

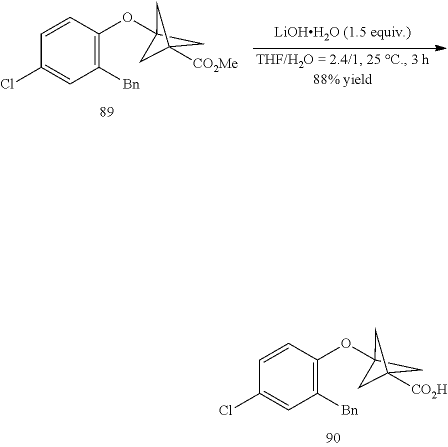 [SMILES: COC(=O)C12CC(Oc3ccc(Cl)cc3Cc3ccccc3)(C1)C2 ; O=C(O)C12CC(Oc3ccc(Cl)cc3Cc3ccccc3)(C1)C2]

A 4 mL borosilicate vial equipped with a magnetic stir bar was charged with 89 (130 mg, 0.379 mmol, 1.00 equiv.) and THE/H₂O=2.4/1 (640 μL, c=0.594 M). The mixture was cooled to 0° C., and LiOH·H₂O (24 mg, 0.57 mmol, 1.5 equiv.) was then added. The resulting reaction mixture was stirred at 25° C. for 3 h. Then, water (5 mL) was added to the mixture, which was then extracted with EtOAc (2×3 mL). The aqueous layer was acidified to PH=1-2 with 1M HCl solution and extracted with EtOAc (3×10 mL). The combined organic phase was dried over Na₂SO₄, filtered, and the solvent was removed under reduced pressure to afford the title compound 90 as a colorless oil (110 mg, 0.335 mmol, 88%).

$${R}_{f} = 0.56 ( pentane / EtOAc , 1 : 1 ( v / v ) ) .$$

HRMS-EI (m/z) calculated for C₁₉H₁₇ClO₃⁺ [M]⁺, 328.0862; found, 328.0861; deviation: −0.4 ppm.

### Bicyclo[1.1.1]pentylamine 91

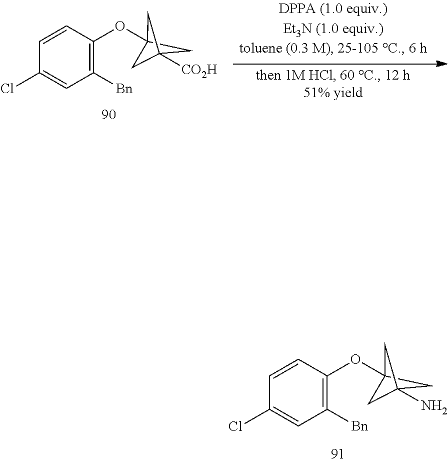 [SMILES: O=C(O)C12CC(Oc3ccc(Cl)cc3Cc3ccccc3)(C1)C2 ; NC12CC(Oc3ccc(Cl)cc3Cc3ccccc3)(C1)C2]

Under argon, to a 5 mL round-bottom flask equipped with a magnetic stir bar were added 90 (32.8 mg, 0.100 mmol, 1.00 equiv.) and anhydrous toluene (0.33 mL, c=0.30 M). Then, Et₃N (14 μL, 10 mg, 0.10 mmol, 1.0 equiv.) and diphenylphosphorylazide (DPPA) (22 μL, 28 mg, 0.10 mmol, 1.0 equiv.) were added at 25° C. The resulting solution was stirred at 25° C. for 3 h and then at 110° C. for 3 h, to form the corresponding isocyanate intermediate. The reaction mixture was then cooled to 60° C., and 1M HCl (0.17 mL) was added. After 12 h, the reaction mixture was cooled to 25° C. and diluted with EtOAc (5 mL). The organic layer was separated and extracted with 1M HCl (3×5 mL). The combined aqueous extracts were basified to pH=12-13 by adding 1M NaOH and extracted with DCM (3×10 mL). The combined organic phase was dried over Na₂SO₄, filtered, and the solvent was removed under reduced pressure. The residue was purified by fast chromatography on silica gel eluting with DCM/MeOH/Et₃N (1/0/0.01-50/1/0.5 (v/v)) to afford the title compound 91 as a yellow oil (15.3 mg, 51.0 μmol, 51%).

HRMS-ESI (m/z) calculated for C₁₈H₁₉NClO⁺ [M+H]⁺, 300.1151; found, 300.1150; deviation: −0.3 ppm.

As discussed above, the incorporation of three-dimensional small-ring scaffolds into bioactive molecules can improve their pharmacokinetic profile, including increased metabolic stability and solubility. Therefore, the rigid 1,3-disubstituted bicyclo[1.1.1]pentanes (BCPs) have shown promise as linear bioisosteres for para-substituted benzene rings in drug development. Construction of BCPs commonly requires the cumbersome use of a volatile and labile [1.1.1]propellane solution as reagent, and more stable reagents do not show the versatile reactivity of propellane itself. The lack of practical reagents for BCP introduction currently impedes the potential of this promising scaffold for pharmaceutical development. Here, the present inventors report stable thianthrenium-based BCP reagents for practical oxygen-, nitrogen-, and carbon alkylation reactions that expand the bicyclopentylation scope of any other reagent, including [1.1.1]propellane; aryl BCP ethers are characterized as a new structural motif in chemistry.

The redox and stereoelectronic properties of the thianthrene scaffold are relevant for both the synthesis via strain release as well as the subsequent reactivity of the new reagents. The weak carbon-sulfur bond can undergo selective mesolytic cleavage upon single-electron reduction to produce BCP radicals that engage in transition-metal-mediated bond formations, including alkylation of phenols, N-heterocycles, amines, amides, sulfonamides, anilines, arenes, and heteroarenes, even at a late-stage with a wide variety of functional groups present. Because the versatile BCP thianthrenium reagents can be prepared centrally and are stable to storage and transport in ambient conditions, they display potential for practical applications in pharmaceutical research for exploration of high-value structures that otherwise may not have been accessed.

1. A thianthrene derivative of the Formula (I): 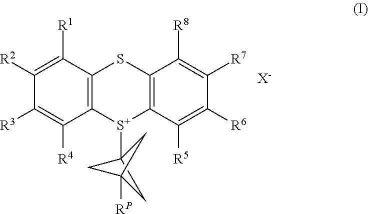
wherein R¹ to R⁸ may be the same or different and are each selected from hydrogen, halogen, a C₁ to C₆ alkyl group, which is optionally substituted by at least one halogen, or a —O—C₁ to C₆ alkyl group, wherein R^{P} represents CF₃ or ON and wherein X⁻ is an anion, selected from F⁻, Cl⁻, triflate⁻, BF₄⁻, SbF₆⁻, PF₆⁻, ClO₄⁻, 0.5 SO₄²⁻ or NO₃.

2. A thianthrene derivative of the Formula (I) as claimed in claim 1 wherein, in Formula (I), R¹ to R⁸ may be the same or different and are each selected from hydrogen, Cl or F, R^{P} is as defined in claim 1 and X⁻ is an anion as defined in claim 1.

3. A thianthrene derivative of the Formula (I) as claimed in claim 1 wherein, in Formula (I), R¹ to R⁸ are each hydrogen, R^{P} is as defined in claim 1 and X⁻ is an anion as defined in claim 1.

4. A thianthrene derivative of the Formula (I) as claimed in claim 1 wherein, in Formula (I), R¹ to R⁸ are each hydrogen, R^{P} is as defined in claim 1 and X⁻ is an anion selected from triflate or BF₄⁻.

5. For a process comprising transferring a bicyclopentyl group to an organic compound selected from phenols, nucleophiles and aryl halides in the presence of a transfer agent, wherein the transfer agent is a bicyclopentyl thianthrenium compound of the Formula (I) as claimed in claim 1.

6. A process comprising transferring a bicyclopentyl group in the presence of a photocatalyst and a transfer agent in a transition-metal-mediated bond formation to an organic compound selected from aryl halides, phenols and nucleophilic compounds, wherein the transfer agent is a bicyclopentyl thianthrenium compound of the Formula (I) as claimed in claim 1.

7. The process according to claim 6, wherein the organic compound is an N nucleophile.

8. The process according to claim 6, wherein the organic compound is substituted by at least one group selected from hydroxyl, aldehyde, carboxylic acid ester, olefin, amino, amido, sulfonamido, halogen selected from bromo, fluoro, and chloro.
